(19) United States
(12) Patent Application Publication
(10) Pub. No.: US 2011/0294138 A1
(43) Pub. Date: Dec. 1, 2011

(54) METHODS FOR SIMULTANEOUSLY MEASURING THE IN VIVO METABOLISM OF TWO OR MORE ISOFORMS OF A BIOMOLECULE

(75) Inventors: Randall J. Bateman, St. Louis, MO (US); Kristin R. Wildsmith, St. Louis, MO (US); Justyna Dobrowolska, St. Louis, MO (US); Ling Munsell, St. Louis, MO (US); Kwasi G. Mawuenyega, St. Louis, MO (US)

(73) Assignee: THE WASHINGTON UNIVERSITY, St. Louis, MO (US)

(21) Appl. No.: 13/129,036

(22) PCT Filed: Nov. 12, 2009
(86) PCT No.: PCT/US09/64146

§ 371 Date: Aug. 1, 2011

Related U.S. Application Data
(60) Provisional application No. 61/113,940, filed on Nov. 12, 2008.
(60) Provisional application No. 61/115,138, filed on Nov. 17, 2008.

(51) Int. Cl. G01N 33/566; C12Q 1/37

(52) U.S. Cl. 435/7.1; 435/23

(57) ABSTRACT

The present invention encompasses methods for the simultaneous measurement of the in vivo metabolism of two or more isoforms of a biomolecule. The biomolecule is typically produced in the central nervous system.

### GOVERNMENT SUPPORT

The present invention was made, at least in part, with funding from the National Institutes of Health Grant No. K23 AG030946. Accordingly, the United States Government may have certain rights in this invention.

### FIELD OF THE INVENTION

The present invention encompasses methods for the simultaneous measurement of the in vivo metabolism of two or more isoforms of a biomolecule.

### BACKGROUND OF THE INVENTION

Alzheimer's Disease (AD) is the most common cause of dementia and is an increasing public health problem. AD, like other central nervous system (CNS) degenerative diseases, is characterized by disturbances in biomolecule production, accumulation, and clearance. As a result, there is a need for efficient methods of measuring biomolecule metabolism in a subject. In particular, there is a need for simultaneously measuring different isomers of a biomolecule in the same sample. Such methods would save time, money, and require fewer samples from a given subject.

### SUMMARY OF THE INVENTION

One aspect of the invention encompasses a method for simultaneously measuring the in vivo metabolism of two or more isoforms of a biomolecule produced in the central nervous system of a subject. The method comprises administering a labeled moiety to the subject. The labeled moiety is typically incorporated into the biomolecule as the biomolecule is produced in the subject. A sample is then obtained from the subject, where the sample comprises a first biomolecule fraction labeled with the moiety, and a second biomolecule fraction not labeled with the moiety. The first biomolecule fraction comprises two or more labeled isoforms of the biomolecule, and the second biomolecule fraction comprises two or more unlabeled isoforms of the biomolecule. The amount of each labeled isoform and the amount of each unlabeled isoform is detected, wherein the ratio of labeled isoform to unlabeled isoform for a particular isoform is directly proportional to the metabolism of the particular isoform in the subject.

Another aspect of the invention encompasses a method for determining whether a therapeutic agent affects the in vivo metabolism of two or more isoforms of a biomolecule produced in the central nervous system of a subject. The method comprises administering the therapeutic agent to the subject and administering a labeled moiety to the subject. The labeled moiety is typically incorporated into the biomolecule as the biomolecule is produced in the subject. A sample is obtained from the subject, and generally comprises a first biomolecule fraction labeled with the moiety, and a second biomolecule fraction not labeled with the moiety. The first biomolecule fraction comprises two or more labeled isoforms of the biomolecule, and the second biomolecule fraction comprises two or more unlabeled isoforms of the biomolecule. The amount of each labeled isoform and the amount of each unlabeled isoform in the biological sample is detected, wherein the ratio of labeled isoform to unlabeled isoform for a particular isoform is directly proportional to the metabolism of the particular isoform in the subject. The metabolism of each isoform is compared to a suitable control value, such that a change from the control value for a particular isoform indicates the therapeutic agent affects the metabolism of the particular isoform in the central nervous system of the subject.

Other aspects and iterations of the invention are described more thoroughly below.

### DESCRIPTION OF THE FIGURES

FIG. 1 illustrates the detection of human ApoE isoform specific tryptic peptides in immortalized knock-in murine astrocytes expressing human ApoE2 or ApoE4 by nano-LC tandem MS. ApoE4 media (0.5 mL) and ApoE2 media (1 mL) were pooled and incubated with 0.1 mL of PHM-Liposorb™ for 30 min at 4° C. The samples were denatured, reduced, and alkylated, digested with trypsin to generate isoform specific peptides, which were analyzed by nano-LC tandem MS. (A) Extracted ion chromatographs for the doubly-charged ion of each isoform specific peptide. (B) Corresponding MS² spectra for each peptide with the b and y ions labeled.

FIG. 2 illustrates the detection of human ApoE isoform specific tryptic peptides in CSF by nano-LC tandem MS. CSF (0.1 mL) from young normal control participants was incubated with 0.1 mL of PHM-Liposorb™ for 30 min at 4° C. The samples were denatured, reduced, and alkylated, digested with trypsin to generate isoform specific peptides, which were analyzed by nano-LC tandem MS. (A) and (B) are derived from an individual with ApoE3/4 genotype. Extracted ion chromatographs are shown in (A) for the doubly-charged ion of each isoform specific peptide, and the corresponding MS² spectra are displayed in (B). (C) and (D) are derived from an individual with ApoE3/2 genotype. Extracted ion chromatographs are shown in (C) for the doubly-charged ion of each isoform specific peptide, and the corresponding MS² spectra are displayed in (D).

FIG. 3 illustrates the detection of ApoE3 specific peptides. ApoE proteins were immunoprecipitated with WUE4 antibody, denatured, reduced, alkylated, and digested with trypsin to generate isoform specific peptides, which were analyzed by nano-LC tandem MS. (A) Extracted ion chromatographs for the ApoE3 specific peptides. (B) Corresponding MS² spectra for each peptide.

FIG. 4 presents standard curves for each of the ApoE isoform specific tryptic peptides isolated from ¹³C-labeled ApoE4 and ApoE2 astrocyte media. Labeled and unlabeled ApoE isoform specific peptides were detected by tandem MS and the percent label calculated from the ratio of the labeled to unlabeled ions. (A) ApoE3/2 peptide. (B) ApoE3/4 peptide. (C) ApoE2 peptide. (D) ApoE4 peptide.

FIG. 5 presents the production of human ApoE4 in immortalized knock-in murine astrocytes. ApoE4 expressing astrocytes were grown to confluency. ¹³C₆-Leucine was added to the media at time t=0 bringing the total percent ¹³C₆-leucine to 50%. Media was collected over 48 hours. ApoE was captured using liposorb, reduced and alkylated, and digested with trypsin. ApoE4 specific peptides were analyzed by nano-LC MS/MS. The SILT method was used to determine the incorporation of ¹³C₆-leucine into ApoE (n=3, error bars SEM). The fractional synthetic rate (FSR) of ApoE4 was calculated using initial slope and plateau tracer to tracee ratio (TTR) of 8.5% per hour.

FIG. 6 presents the metabolism of ApoE isoforms in human CNS. ApoE2, ApoE3, and ApoE4 were isolated from CSF of young normal control participants infused with ¹³C₆-leucine (2 mg/kg/h) from 0-9 h. (A) ApoE4=LGADMEDVR (SEQ ID NO:3), ApoE3=LGADMEDVcGR (SEQ ID NO:1). (B) ApoE3=LAVYQAGAR (SEQ ID NO:2), ApoE2=cLAVYQAGAR (SEQ ID NO:4). TTR=tracer to tracee ratio.

FIG. 7 illustrates the detection of soluble APP-beta in CHO media. (A). Depicts the separation of unlabeled and labeled soluble APP-beta via nano-LC. Both peptides elute at the same time, 23.34 min. (B) Shows paired spectra of unlabeled and labeled soluble APP-beta peptides. The spectra look similar except for small shifts in the masses of the ions designated with arrows. The designated ions are 3 m/z heavier in the labeled sAPP-beta peptide because these ions are doubly charged. Comparison of the signal intensity of the labeled ion to unlabeled ion give the % labeled to % unlabeled ratio of the ion. The ratios of many ions are summated to give an accurate % labeled/% unlabeled ratio for the whole sAPP-beta peptide.

FIG. 8 illustrates the detection of soluble APP-alpha in CHO media. (A) Depicts the elution of unlabeled and labeled soluble APP-alpha via nano-LC. Both peptides elute at the same time, 25.18 min. (B) Shows paired spectra of unlabeled and labeled soluble APP-alpha peptides. The designated labeled ions are 2 m/z heavier in the labeled ions because these ions are triply charged. Comparison of the signal intensity of the labeled ion to unlabeled ion give the % labeled to % unlabeled ratio of the ion. The ratios of many ions are summated to give an accurate % labeled/% unlabeled ratio for the whole sAPP-beta peptide.

FIG. 9 presents standard curves for each of the sAPP-alpha and sAPP-beta peptides. The actual labeled to unlabeled ratio detected and quantitated is plotted against the theoretical percent labeled to unlabeled (based on the known labeled to unlabeled leucine of CHO media). (A) sAPP-alpha peptide. (B) sAPP-beta peptide.

FIG. 10 depicts two graphs illustrating the change in ratio of the percent labeled to percent unlabeled soluble APP ions over 48 hours in a human subject as measured in CSF. (A) represents soluble APP beta peptide ions. (B) represents soluble APP alpha peptide ions. The production and clearance rates of soluble APP alpha and soluble APP beta can be determined by calculating the production and clearance rates of the summated APP alpha or APP beta peptide ions.

FIG. 11 illustrates the detection of amyloid-beta₂₉₋₄₀ peptide from a mixture of amyloid-beta₂₉₋₄₀, amyloid-beta₂₉₋₄₂, amyloid-beta₂₉₋₃₈, etc. Amyloid-beta was immunoprecipitated with an anti-amyloid-beta antibody, and digested with trypsin. The peptide was analyzed by nano-LC tandem MS. (A) Extracted ion chromatographs for the amyloid-beta₂₉₋₄₀ peptide. (B) Corresponding MS² spectra for each peptide.

FIG. 12 depicts graphs illustrating the detection of Aβ isoform specific Lys-N peptides in CSF by nano-LC tandem MS. CSF (1 mL) from young normal control participants was incubated with HJ5.1 antibody at RT for 2 hours. The purified Aβ was digested with Lys-N to generate isoform specific peptides, which were analyzed by nano-LC tandem MS. Extracted ion chromatographs are shown in panels for the doubly-charged ion of each isoform specific peptide: (A) total elution profile of digest, (B) Aβ 1-38, (C) Aβ 1-40, and (D) Aβ 1-42. The corresponding MS² spectra are displayed in the bottom panels: (E) Aβ 1-38, (F) Aβ 1-40, and (G) Aβ 1-42.

FIG. 13 depicts graphs illustrating standard curves for each of the Aβ isoform specific Lys-N peptides isolated from ¹³C-labeled Aβ cell cultured media. Labeled and unlabeled Aβ isoform specific peptides were detected by tandem MS and the percent label calculated from the ratio of the labeled to unlabeled ions. (A) Aβ 1-38. (B) Aβ 1-40. (C) Aβ 1-42.

FIG. 14 depicts graphs illustrating the time course production and clearance of Aβ in a human subject. The individual curves for Aβ 1-40 and Aβ 1-42 were derived from data acquired from a single immunoprecipitation from human CSF.

### DETAILED DESCRIPTION OF THE INVENTION

The present invention provides methods for simultaneously measuring the in vivo metabolism of two or more isoforms of a biomolecule or a protein in the central nervous system (CNS). In particular, the invention provides methods for determining the in vivo production and clearance rates of several isoforms of a biomolecule or a protein of interest in one biological sample. The method of the invention may be used to compare the metabolism of several different isoforms of a CNS-derived biomolecule or protein to determine whether the metabolism differs among the various isoforms or whether the metabolism of one or more of the isoforms changes over time. Typically, the biomolecule or protein of interest is implicated in a neurological or neurodegenerative disease or disorder, such that the methods may be used to diagnose or monitor the progression or treatment of a neurological or neurodegenerative disease or disorder. Furthermore, methods are provided to determine whether a therapeutic agent affects the in vivo metabolism of several different isoforms of a CNS-derived biomolecule or protein.

### (I) Methods for Simultaneously Measuring the Metabolism of Several Isoforms

The present invention provides methods for concurrently measuring the in vivo metabolism of two or more isoforms of a CNS derived biomolecule or protein in a single biological sample. The CNS derived biomolecule or protein may be implicated in a neurological or neurodegenerative disease or disorder. In particular, the method comprises labeling the biomolecule or protein of interest as it is being produced in the CNS, collecting a biological sample comprising labeled and unlabeled isoforms of the biomolecule or protein, and quantitating the amount of each labeled and unlabeled isoform such that the metabolism of each isoform may be determined. This method may be used to calculate metabolic parameters, such as the production and clearance rates within the CNS, for each of the two or more isoforms of the biomolecule or protein of interest.

### (a) Neurodegenerative Diseases

Those of skill in the art will appreciate that the method of the invention may be used to determine the metabolism of several different isoforms of CNS derived biomolecules or proteins implicated in several neurological and/or neurodegenerative diseases, disorders, or processes, Non-limiting examples of suitable diseases or disorders include Alzheimer's Disease, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases (e.g. Creutzfeldt-Jakob Disease, bovine spongiform encephalopathy or Mad Cow Disease, and scrapie), Lewy Body Disease, schizophrenia, and Amyotrophic Lateral Sclerosis (ALS or Lou Gehrig's Disease). It is also envisioned that the method of the invention may be used to study the normal physiology, metabolism, and function of the CNS.

The in vivo metabolism of isoforms of CNS derived biomolecules or proteins may be measured in mammalian subjects. In one embodiment, the subject may be a companion animal such as a dog or cat. In another embodiment, the subject may be a livestock animal such as a cow, pig, horse, sheep or goat. In yet another embodiment, the subject may be a zoo animal. In another embodiment, the subject may be a research animal such as a non-human primate or a rodent. In yet another embodiment, the subject may be a human. The subject may or may not be afflicted with, or pre-disposed to, a neurological or neurodegenerative disease or disorder listed above.

### (b) Isoforms of CNS Derived Biomolecules or Proteins

The present invention provides a method for measuring the metabolism of two or more isoforms of a biomolecule or protein produced in the CNS. The biomolecule may be a protein, a lipid, a nucleic acid, or a carbohydrate. The possible biomolecules are only limited by the ability to label them during in vivo production or processing and collect a sample or samples from which their metabolism may be measured. In exemplary embodiments, the biomolecule is a protein. Non-limiting example of suitable proteins include amyloid-β (Aβ) and its C-terminal and N-terminal variants, amyloid precursor protein (APP), apolipoprotein E (isoforms 2, 3, or 4), apolipoprotein J (also called clusterin), Tau (another protein associated with AD), phospho Tau, glial fibrillary acidic protein, alpha-2 macroglobulin, synuclein, S100B, Myelin Basic Protein (implicated in multiple sclerosis), prions, interleukins, TDP-43, superoxide dismutase-1, huntingtin, tumor necrosis factor (TNF), heat shock protein 90 (HSP90), and combinations thereof. Additional biomolecules that may be targeted include products of, or proteins or peptides that interact with, GABAergic neurons, noradrenergic neurons, histaminergic neurons, seratonergic neurons, dopaminergic neurons, cholinergic neurons, and glutaminergic neurons.

The term “isoform” refers to different forms of a biomolecule or a protein. The different forms of the biomolecule or protein may be produced by a variety of processes or mechanisms. In embodiments in which the biomolecule is a protein, the isoforms may be proteins that differ in sequence by one or more amino acids. For example, the protein isoforms may be genetic alleles. Alternatively, the protein isoforms may be the products of alternate splicing, RNA editing, posttranslational processing, and the like. As detailed below, such isoforms may be analyzed intact or they may be detected via ex vivo cleavage of the various forms of the protein to generate “isoform specific fragments.”

In other embodiments in which the biomolecule is a protein, the isoforms may be cleavage products that are generated in vivo by protease activity. Non-limiting examples of suitable in vivo proteases include alpha-secretase, beta-secretase, and variable gamma-secretase. Alternatively, the cleavage products may also be generated in vivo by non-proteases. Suitable non-protease cleavage systems include, but are not limited to, lysosomal enzymes, superoxide dismutases, free radicals, and the like.

In one preferred embodiment, the protein may be amyloid precursor protein (APP) and the isoforms of APP may be soluble APP-alpha and soluble APP-beta, which are cleavage products of alpha-secretase and beta-secretase, respectively. In another preferred embodiment, the protein may be amyloid-beta and the C-terminal isoforms of amyloid-beta may be amyloid-beta₁₋₃₇, amyloid-beta₁₋₃₈, amyloid-beta₁₋₃₉, amyloid-beta₁₋₄₀, amyloid-beta₁₋₄₁, amyloid-beta₁₋₄₂, and/or amyloid-beta₁₋₄₃. In still another preferred embodiment, the protein may be apolipoprotein E (ApoE) and the isoforms of ApoE may be isoform specific fragments of genetic isoforms of ApoE (e.g., ApoE2, ApoE3, and ApoE4).

### (c) Labeled Moiety

The labeled moiety may comprise a radioactive isotope or a non-radioactive (stable) isotope. In a preferred embodiment, non-radioactive isotopes may be used and measured by mass spectrometry. Preferred stable isotopes include deuterium (²H), ¹³C, ¹⁵N, ¹⁷O, ¹⁸O, ³³S, ³⁴S, or ³⁶S, but it is recognized that a number of other stable isotope that change the mass of an atom by more or less neutrons than is seen in the prevalent native form would also be effective. A suitable label generally will change the mass of the fragment of the isoform under study such that it can be detected in a mass spectrometer. In one embodiment, the biomolecule to be measured may be a nucleic acid, and the labeled moiety may be a nucleoside triphosphate comprising a non-radioactive isotope (e.g., ¹⁵N). In another embodiment, the biomolecule to be measured may be a protein, and the labeled moiety may be an amino acid comprising a non-radioactive isotope (e.g., ¹³C). Alternatively, a protein may be labeled post-translationally with a labeled moiety such as acetate or ATP comprising a suitable isotope.

In a preferred embodiment, when the method is employed to measure the metabolism of protein isoforms, the labeled moiety typically will be an amino acid. Those of skill in the art will appreciate that several amino acids may be used in the method of the invention. Generally, the choice of amino acid is based on a variety of factors such as: (1) At least one residue of the amino acid is present in each of the two or more isoforms of the protein of interest. (2) The amino acid is generally able to rapidly equilibrate across the blood-brain barrier and quickly reach the site of protein production. Leucine is a preferred amino acid to label proteins that are produced in the CNS, as demonstrated in the examples. (3) The amino acid ideally may be an essential amino acid (not produced by the body), so that a higher percent of labeling may be achieved. Non-essential amino acids may also be used; however, measurements will likely be less accurate. (4) The amino acid label generally does not influence the metabolism of the protein of interest (e.g., very large doses of leucine may affect muscle metabolism). And (5) availability of the desired amino acid (i.e., some amino acids are much more expensive or harder to manufacture than others). In one preferred embodiment, the labeled amino acid may be ¹³C₆-phenylalanine, which contains six ¹³C atoms. In another preferred embodiment, the labeled amino acid may be ¹³C₆-leucine. It is also envisioned that one or more labeled amino acids comprising different stable isotopes may be used simultaneously or in sequence without departing from the scope of the invention.

There are numerous commercial sources of labeled amino acids, both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made with known synthetic chemical processes.

### (d) Administration of the Labeled Moiety

The labeled moiety may be administered to a subject by several methods. Suitable routes of administration include intravenously, intra-arterially, subcutaneously, intraperitoneally, intramuscularly, or orally. In preferred embodiments in which proteins are labeled, the labeled moiety may be an amino acid. In one embodiment, the labeled amino acid may be administered by intravenous infusion. In another embodiment, the labeled amino acid may be orally ingested.

In preferred embodiments, the labeled moiety may be administered 1) slowly over a period of time, 2) as a large single dose depending upon the type of analysis chosen (e.g., steady state or bolus/chase), or 3) slowly over a period of time after an initial bolus dose. To achieve steady-state levels of the labeled biomolecule or protein, the labeling time generally should be of sufficient duration so that the labeled biomolecule or protein may be reliably quantified. In one embodiment, the labeled amino acid may be labeled leucine and the labeled leucine may be administered intravenously. The labeled leucine may be administered intravenously for a period of time ranging from about one hour to about 24 hours. The rate of administration of labeled leucine may range from about 0.5 mg/kg/hr to about 5 mg/kg/hr, preferably from about 1 mg/kg/hr to about 3 mg/kg/hr, or more preferably from 1.8 mg/kg/hr to about 2.5 mg/kg/hr. In another embodiment, the labeled leucine may be administered as a bolus of between about 50 and about 500 mg/kg body weight of the subject, between about 50 and about 300 mg/kg body weight of the subject, or between about 100 and about 300 mg/kg body weight of the subject. In yet another embodiment, the labeled leucine may be administered as a bolus of about 200 mg/kg body weight of the subject. In an alternate embodiment, the labeled leucine may be administered intravenously as detailed above after an initial bolus of between about 0.5 to about 10 mg/kg, between about 1 to about 4 mg/kg, or about 2 mg/kg body weight of the subject.

Those of skill in the art will appreciate that the amount (or dose) of the labeled amino acid can and will vary. Generally, the amount is dependent on (and estimated by) the following factors. (1) The type of analysis desired. For example, to achieve a steady state of about 15% labeled leucine in plasma requires about 2 mg/kg/hr over about 9 hr after an initial bolus of 2 mg/kg over 10 min. In contrast, if no steady state is required, a large bolus of labeled leucine (e.g., 1 or 5 grams of labeled leucine) may be given initially. (2) The protein under analysis. For example, if the protein is being produced rapidly, then less labeling time may be needed and less label may be needed—perhaps as little as 0.5 mg/kg over 1 hour. However, most proteins have half-lives of hours to days and, so more likely, a continuous infusion for 4, 9 or 12 hours may be used at 0.5 mg/kg to 4 mg/kg. And (3) the sensitivity of detection of the label. For example, as the sensitivity of label detection increases, the amount of label that is needed may decrease.

Those of skill in the art will appreciate that more than one labeled amino acid may be used in a single subject. This would allow for multiple labeling of the two or more isoforms of the protein of interest and may provide information on the metabolism of each of the isoforms at different times. For example, a first labeled amino acid may be given to subject over an initial time period, followed by a therapeutic agent, and then a second labeled amino acid may be administered. In general, analysis of the samples obtained from this subject would provide a measurement of metabolism of each of the protein isoforms before AND after administration of the therapeutic agent, thereby directly measuring the pharmacodynamic effect of the therapeutic agent in the same subject. Alternatively, multiple labeled amino acids may be administered to the subject at the same time to increase the labeling of the multiple isoforms of the protein of interest.

### (e) Biological Sample

The method of the invention provides that a biological sample be obtained from the subject such that the in vivo metabolism of the two or more isoforms of the biomolecule or protein of interest may be determined. The biological sample comprising labeled and unlabeled isoforms of interest that is collected for analysis can and will vary depending upon the embodiment. In some embodiments, the biological sample may be a body fluid. Suitable body fluids include, but are not limited to, cerebral spinal fluid (CSF), blood plasma, blood serum, whole blood, urine, saliva, perspiration, and tears. In other embodiments, the biological sample may be a CNS tissue, i.e., a brain tissue or a spinal cord tissue. The biological sample generally will be collected using standard procedures well known to those of skill in the art.

For example, cerebrospinal fluid may be obtained by lumbar puncture with or without an indwelling CSF catheter (a catheter is preferred if multiple collections are made over time). Blood may be collected by veni-puncture with or without an intravenous catheter. Urine may be collected by simple urine collection or more accurately with a catheter. Saliva and tears may be collected by direct collection using standard good clinical practice (GCP) methods. CNS tissue may be obtained via biopsy, dissection, or resection, or, alternatively, it may be obtained using laser microdissection. The subject may or may not have to be sacrificed to obtain the CNS tissue sample, depending on the CNS sample desired and the subject utilized.

In general, the invention typically provides that a first biological sample be taken from the subject prior to administration of the labeled moiety to provide a baseline for the subject. After administration of the labeled moiety, one or more biological samples generally would be taken from the subject. As will be appreciated by those of skill in the art, the number of samples and when they would be taken generally will depend upon a number of factors such as: the type of analysis, type of administration, the protein of interest, the rate of metabolism, the type of detection, etc.

In one embodiment, biological samples (e.g., blood and/or CSF) may be taken hourly for 36 hours after start of the labeling. Alternatively, samples may be taken every other hour or even less frequently. In general, biological samples obtained during the first few half-lives of the protein (e.g., about 12 hrs after the start of labeling for amyloid-beta) may be used to determine the rate of production of each of the isoforms, and biological samples taken several half-lives of the protein after the labeling has been terminated (e.g., about 24-36 hrs after the start of labeling for amyloid-beta) may be used to determine the clearance rate of each of the isoforms. In another alternative, one sample may be taken after labeling for a period of time, such as 12 hours, to estimate the production rate, but this may be less accurate than multiple samples. In yet a further alternative, samples may be taken from an hour to days or even weeks apart depending upon the production and clearance rates of the protein.

### (f) Detection

The method further comprises detecting the amount of each labeled isoform and each unlabeled isoform of the biomolecule or protein of interest such that the in vivo metabolism of the different isoforms may be determined. The ratio of labeled isoform to unlabeled isoform is directly proportional to the metabolism of that isoform in the CNS of the subject. Suitable methods for the detection of labeled and unlabeled isoforms can and will vary depending upon the biomolecule or protein of interest and the type of labeled moiety used to label the biomolecule or protein of interest.

In a preferred embodiment, mass spectrometry may be used to detect differences in mass between the labeled and unlabeled isoforms of the biomolecule or protein. In one embodiment, gas chromatography mass spectrometry may be used to detect the amounts of labeled and unlabeled isoforms. In an alternate embodiment, MALDI-TOF mass spectrometry may be used to detect the labeled and unlabeled isoforms. In a preferred embodiment, high-resolution tandem mass spectrometry may be used to detect the labeled and unlabeled isoforms.

In exemplary embodiments, the biomolecule is a protein and different isoforms of the protein are detected by mass spectrometry. Those of skill in the art will appreciate that isoforms corresponding to in vivo-generated cleavage products may be detected as is, or they may be further digested in vitro to generate smaller peptides for detection by mass spectrometry. Isoforms corresponding to different forms of a protein (i.e., genetic alleles, post-transcriptional, translational, or post-translational processes) are generally digested ex vivo to generate isoform specific fragments for detection by mass spectrometry. “Isoform specific fragments” are typically generated by isolating the proteins of interest (see below) and digesting them with a suitable protease (as detailed in Example 1). Non-limiting examples of suitable proteases that may be used to generate isoform specific fragments include trypsin, chymotrypsin, endopeptidase Arg-C, endopeptidase Lys-C, and endopeptidase Glu-C.

Additional techniques may be utilized to isolate the isoforms of a biomolecule or protein from other biomolecules in the biological sample before detection and analysis. As an example, the labeled and unlabeled isoforms may be isolated and purified by immunoprecipitation using a specific antibody. In another embodiment, the labeled and unlabeled isoforms may be isolated and purified by adsorption to a derivatized polymer. For example, the polymer may be conjugated with antibodies specific to the isoforms of interest, or the polymer may be conjugated with another substrate that binds the isoforms. Alternatively, the polymer may be a polyhydroxymethylene polymer that is derivatized with a fat oxyethylized alcohol (such as PHM-Liposorb™ produced by Calbiochem, San Diego, Calif.) such that the polymer binds lipoproteins. Furthermore, the isoforms may also be isolated and/or separated by liquid chromatography. For example, mass spectrometers having chromatography setups may be used to isolate and/or separate the isoforms, which are then subjected to mass spectrometry, as demonstrated in the examples.

In one exemplary embodiment, the isoforms may be immunoprecipitated, digested into smaller peptides, and then analyzed by a liquid chromatography system interfaced with a tandem MS unit equipped with an electrospray ionization source (LC-ESI-tandem MS). In another exemplary embodiment, the isoforms interest may be isolated by adsorption to a derivatized PHM matrix, digested into smaller peptides (or isoform specific fragments), and then analyzed by a liquid chromatography system interfaced with a tandem MS unit equipped with an electrospray ionization source (LC-ESI-tandem MS).

### (g) Analysis

Once the amount of the two or more labeled and unlabeled isoforms has been detected in the sample, the ratio or percent of each labeled isoform may be determined. The metabolism (production rate, clearance rate, lag time, half-life, etc.) of each isoform may be calculated from the ratio of labeled to unlabeled isoform over time. There are many suitable ways to calculate these parameters.

The method of the invention allows measurement of the amount of each of the labeled and unlabeled isoforms at the same time in the same sample, such that the ratio of labeled to unlabeled for each isoform may be calculated. Those of skill in the art will be familiar with the first order kinetic models of labeling that may be used with the method of the invention. For example, the fractional synthesis rate (FSR) of each of the isoforms may be calculated. The FSR equals the initial rate of increase of labeled to unlabeled isoform divided by the precursor enrichment. Likewise, the fractional clearance rate (FCR) of each of the isoforms may be calculated. In addition, other parameters (such as, e.g., absolute production rate, absolute clearance rate, area-under-curve analysis of newly generated biomolecules, lag time, and isotopic tracer steady state) may be determined for each of the isoforms and may be used as indicators of the isoform's metabolism and physiology. Also, modeling may be performed on the data to fit multiple compartment models to estimate transfer between compartments. Of course, the type of mathematical modeling chosen will depend on the individual protein production and clearance parameters (e.g., one-pool, multiple pools, steady state, non-steady-state, compartmental modeling, etc.).

The invention provides that the production of the isoforms of interest is typically based upon the rate of increase of the labeled/unlabeled isoform ratio over time (i.e., the slope, the exponential fit curve, or a compartmental model fit defines the rate of production). For these calculations, a minimum of one sample is typically required (one could estimate the baseline label), two are preferred, and multiple samples are more preferred to calculate an accurate curve of the uptake of the label into the biomolecule or protein (i.e., the production rate). Conversely, after the administration of label is terminated, the rate of decrease of the ratio of labeled to unlabeled isoform typically reflects the clearance rate of that isoform. For these calculations, a minimum of one sample is typically required (one could estimate the baseline label), two are preferred, and multiple samples are more preferred to calculate an accurate curve of the decrease of the label from the biomolecule or protein over time (i.e., the clearance rate). The amount of each labeled isoform in a sample at a given time reflects the production rate or the clearance rate (i.e., removal or destruction) and is usually expressed as percent per hour or the mass/time (e.g., mg/hr) of that isoform in the subject.

### (h) Applications

The method of the invention may be used to determine whether there are differences in the in vivo metabolism of the two or more isoforms of the biomolecule or protein of interest. The rate of production or the rate of clearance of one isoform may differ from the rate of production or the rate of clearance of the other isoforms of interest. Furthermore, the method of the invention may be used to determine whether the in vivo metabolism of one or more of the isoforms of interest changes over time. Such information may allow a person of skill in the art to diagnose or monitor the progression or treatment of a neurological or neurodegenerative disease or disorder. Similarly, such information may facilitate an understanding of the etiology and/or pathophysiology of the disease or disorder.

In addition, the method may be used to determine the efficacy of a therapeutic treatment designed to affect various isoforms differentially. For example, a gamma-secretase modulator may be designed to decrease the amyloid-beta₁₋₄₂ isoform, while increasing the amyloid-beta₁₋₃₈ isoform. The method of this invention may be used to measure both amyloid-beta isoforms production and clearance rates and identify specific therapeutic responses to both isoforms in the same sample and experiment. This invention allows for the direct comparison of isoforms in the same sample from the same experiment. This controls for different experimental conditions and removes the necessity of assumptions between control and experimental conditions, thus decreasing the biologic and experimental variability of the experiment. In addition, this invention also decreases the number of sample preparations directly by measuring more than one isoform in each sample preparation. Also, this invention decreases the need for specific purification or antibodies to each protein isoform, as all protein isoforms can be collected together and the LC-MS then can analyze all isoforms together. This will lead to substantial reductions in time to develop specific antibodies for each isoform, sample preparation time, and also mass spectrometry analysis time. The more isoforms analyzed, the greater the time and resource savings will be.

### (II) Methods for Determining Whether a Therapeutic Agent Affects the Metabolism of Two or More Fragments of a Protein

Another aspect of the present invention provides a method for assessing whether a therapeutic agent used to treat a neurological or neurodegenerative disease or disorder affects the metabolism of any of the various isoforms of a biomolecule or protein produced in the CNS of a subject. For example, the metabolism of each of the isoforms may be measured via their respective fragments to determine whether a particular therapeutic agent results in an increase in production, a decrease in production, an increase in clearance, or a decrease in clearance of a given isoform. Accordingly, use of this method will allow those of skill in the art to accurately determine the extent of altered production or clearance of the isoform of interest, and correlate these measurements with the clinical outcome of a treatment. Results from this method, therefore, may help determine the optimal doses and frequency of doses of a therapeutic agent, may assist in the decision-making regarding the design of clinical trials, and may ultimately accelerate validation of effective therapeutic agents for the treatment of neurological or neurodegenerative diseases.

The method comprises administering a therapeutic agent and a labeled moiety to a subject, wherein the label is incorporated into the biomolecule or protein as it is produced in the CNS. In one embodiment, the therapeutic agent may be administered to the subject prior to the administration of the labeled moiety. In another embodiment, the labeled moiety may be administered to the subject prior to the administration of the therapeutic agent. The period of time between the administration of each may be several minutes, an hour, several hours, or many hours. In still another embodiment, the therapeutic agent and the labeled moiety may be administered simultaneously. The method further comprises collecting at least one biological sample comprising labeled and unlabeled isoforms of the biomolecule or protein of interest, detecting the amount of labeled and unlabeled isoforms to determine the metabolism of each isoform, and comparing the metabolism of each isoform to a suitable control value to determine whether the therapeutic agent alters the rate of production or the rate of clearance of a particular isoform in the CNS of the subject.

Non-limiting examples of neurodegenerative diseases, isoforms of biomolecules or proteins, labeled moieties, routes of administration of the labeled moiety, biological samples, means of detection, and means of analysis are detailed above in sections (I)(a), (I)(b), (I)(c), (I)(d), (I)(e), (I)(f), and (I)(g), respectively.

### (a) Therapeutic Agent

Those of skill in the art will appreciate that the therapeutic agent can and will vary depending upon the neurological or neurodegenerative disease or disorder to be treated and/or the isoforms whose metabolism is being analyzed. In embodiments in which the isoforms include soluble APP-alpha, soluble APP-beta, or amyloid-beta (Aβ) peptides, non-limiting examples of suitable therapeutic agents include gamma-secretase inhibitors, gamma-secretase modulators, beta-secretase inhibitors, alpha-secretase activators, RAGE inhibitors, small molecule inhibitors of Aβ production, small molecule inhibitors of Aβ polymerization, platinum-based inhibitors of Aβ production, platinum-based inhibitors of polymerization, agents that interfere with metal-protein interactions, proteins (such as, e.g., low-density lipoprotein receptor-related protein (LRP) or soluble LRP) that bind soluble Aβ, and antibodies that clear soluble Aβ and/or break down deposited Aβ. Other therapeutic agents used to treat Alzheimer's disease include cholesterylester transfer protein (CETP) inhibitors, metalloprotease inhibitors, cholinesterase inhibitors, NMDA receptor antagonists, hormones, neuroprotective agents, and cell death inhibitors. Many of the above mentioned therapeutic agents may also affect the in vivo metabolism of other proteins implicated in neurodegenerative disorders. Additional therapeutic agents that may affect the metabolism of tau and tau isoforms, for example, include tau kinase inhibitors, tau aggregation inhibitors, cathepsin D inhibitors, etc. Furthermore, therapeutic agents that may affect the in vivo metabolism of synuclein and synuclein isoforms include sirtuin 2 inhibitors, synuclein aggregation inhibitors, proteosome inhibitors, etc. Those of skill in the art appreciate that a variety of different therapeutic agents may be utilized in the method of the invention.

The therapeutic agent may be administered to the subject in accord with known methods. Typically, the therapeutic agent will be administered orally, but other routes of administration such as parenteral or topical may also be used. The amount of therapeutic agent that is administered to the subject can and will vary depending upon the type of agent, the subject, and the particular mode of administration. Those skilled in the art will appreciate that dosages may be determined with guidance from Goodman & Goldman's The Pharmacological Basis of Therapeutics, Tenth Edition (2001), Appendix II, pp. 475-493, and the Physicians' Desk Reference.

### (b) Control Value

In general, the control value refers to the in vivo metabolism of the particular isoform of interest in the same subject prior to administration of the therapeutic agent, a control isoform predicted to be differentially affected by the therapeutic agent, or in a different subject who is not administered the therapeutic agent. Differences between the test subject and the control subject generally will reveal whether the therapeutic agent affects the rate of production or the rate of clearance of the particular isoform of interest. A therapeutic agent may differentially alter the metabolism of one isoform of interest, such that this information may be used to predict which subjects will respond to a particular therapeutic agent. Furthermore, this information may be used to determine the appropriate dose and timing of administration of a particular therapeutic agent.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Aβ” refers to amyloid beta.

“Clearance rate” refers to the rate at which a biomolecule or isoform thereof is removed.

“CNS sample” refers to a biological sample derived from a CNS tissue or a CNS fluid. “CNS tissue” includes all tissues within the blood-brain barrier. Similarly, a “CNS fluid” includes all fluids within the blood-brain barrier.

“CNS derived cells” includes all cells within the blood-brain-barrier including neurons, astrocytes, microglia, choroid plexus cells, ependymal cells, other glial cells, etc.

“Fractional clearance rate” or FCR is calculated as the natural log of the ratio of a labeled biomolecule or isoform thereof over a specified period of time.

“Fractional synthesis rate” or FSR is calculated as the slope of the increasing ratio of a labeled biomolecule or isoform thereof over a specified period of time divided by the predicted steady state value of the labeled precursor.

“Isoform” refers to any alternative form of a biomolecule or a protein. Isoforms of a protein may be generated by the following non-limiting mechanisms: different genetic alleles or copies, alternative splicing of a protein during transcription, altered processing during translation, post-translational modifications, post-production modifications, endogenous processing, or endogenous cleavage into products.

“Isotope” refers to all forms of a given element whose nuclei have the same atomic number but have different mass numbers because they contain different numbers of neutrons. By way of a non-limiting example, ¹²C and ¹³C are both stable isotopes of carbon.

“Lag time” generally refers to the delay of time from when a biomolecule or isoform thereof is first labeled until the labeled biomolecule or isoform thereof is detected.

“Metabolism” refers to any combination of the production, transport, breakdown, modification, or clearance rate of a biomolecule or isoform thereof.

“Production rate” refers to the rate at which a biomolecule or isoform thereof is produced.

“Steady state” refers to a state during which there is insignificant change in the measured parameter over a specified period of time.

In metabolic tracer studies, a “stable isotope” is a nonradioactive isotope that is less abundant than the most abundant naturally occurring isotope.

“Subject” as used herein means a living organism having a central nervous system. In particular, the subject may be a mammal. Suitable subjects include research animals, companion animals, farm animals, and zoo animals.

All of the methods disclosed and claimed herein can be performed and executed without undue experimentation in light of the present disclosure. While the methods of this invention have been described in terms of preferred embodiments, it will be apparent to those of skill in the art that variations may be applied to the methods or in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the claims.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Apolipoprotein E Isoform Stable Isotope Labeling Kinetics

Apoliprotein E (ApoE) is a major risk factor for Alzheimer's disease. Humans have three major alleles resulting in ApoE isoforms: ApoE2 (cys112, cys158), ApoE3 (cys112, arg158), and ApoE4 (arg112, arg158). The following example demonstrates a method for the simultaneous detection and quantitation of the different ApoE isoforms in the same sample using stable isotope-labeling tandem mass spectrometry.

The sources of ApoE were ¹³C-labeled and unlabeled biological samples. Labeled and unlabeled human CSF samples were obtained from on-going in vivo stable isotope-labeling studies. Astrocyte media was collected from immortalized mouse astrocytes derived from knock-in mice expressing human ApoE2 or ApoE4. Cells were grown to near confluency in serum containing growth media, at which time the media was changed to serum free media supplemented with 0, 1.25, 2.5, 5, 10, or 20% ¹³C₆-leucine (98% ¹³C₆, Cambridge Isotope Laboratories, Andover, Mass.) for 24 hours.

ApoE was isolated either by overnight incubation with anti-ApoE antibody (WUE4) coupled to CNBr-activated Sepharose beads, or incubation for 30 min (4° C.) with PHM-Liposorb™ (Calbiochem, San Diego, Calif.). Beads were washed with PBS and 100 mM Tris, pH 8.5. The bound ApoE protein was denatured using trifluoroethanol (40% on 25 mM triammonium bicarbonate). After denaturation, ApoE protein was reduced with 5 mM dithiothreitol (DTT) for 30 min at room temperature and alkylated with 10 mM iodoacetamine (IAM) for 30 min at room temperature in the dark. The samples were digested, on-bead, with 0.5 μg of trypsin at 37° C. overnight. After desalting using Nu-tip carbon tips (Glygen Corp., Columbia, Md.), the supernatant containing ApoE peptides was analyzed by nano-LC tandem MS (Thermo-Finnigan LTQ equipped with a New Objective nanoflow ESI source) as previously described (Bateman et al., 2006, Nature Medicine, 12:856-861). The samples were quantitated using the stable-isotope tandem labeled (SILT) mass spectroscopy method as described by Bateman et al. 2007 J Am Soc Mass Spectrum 18(6):007-1006. The labeled ion mass will be shifted by 6, 3, 2, or 1.5 m/z depending on the charge of any specific precursor (i.e., +1, +21, +3, or +4, respectively). The percentage of labeled ApoE was calculated using the ratio of all the b- and y-ions of the labeled to unlabeled peptides.

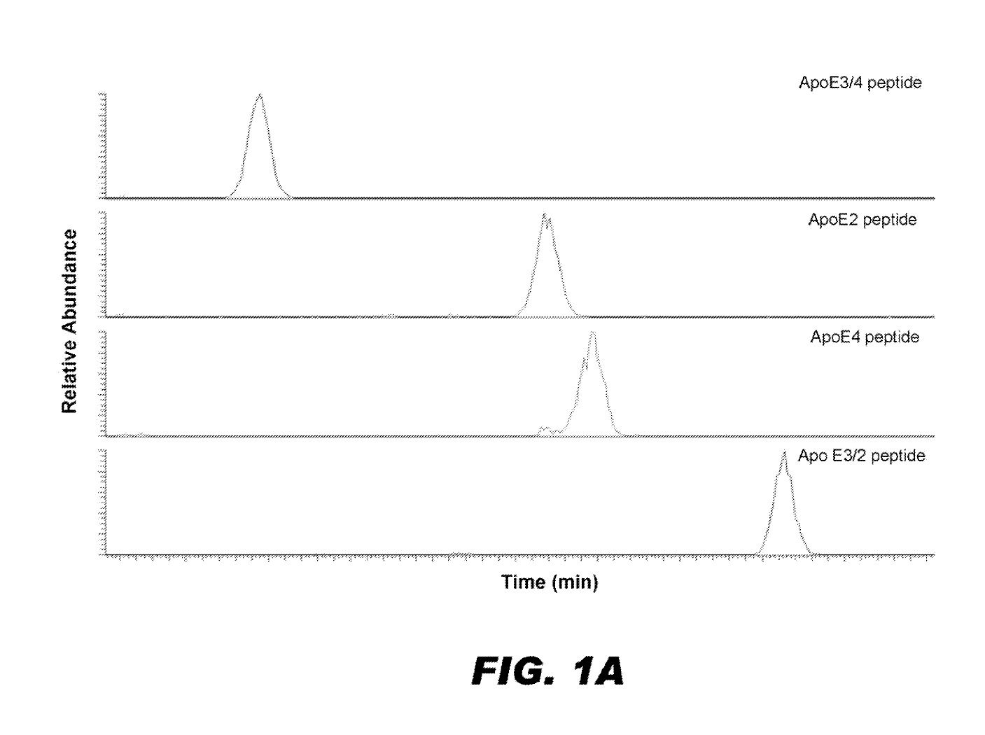

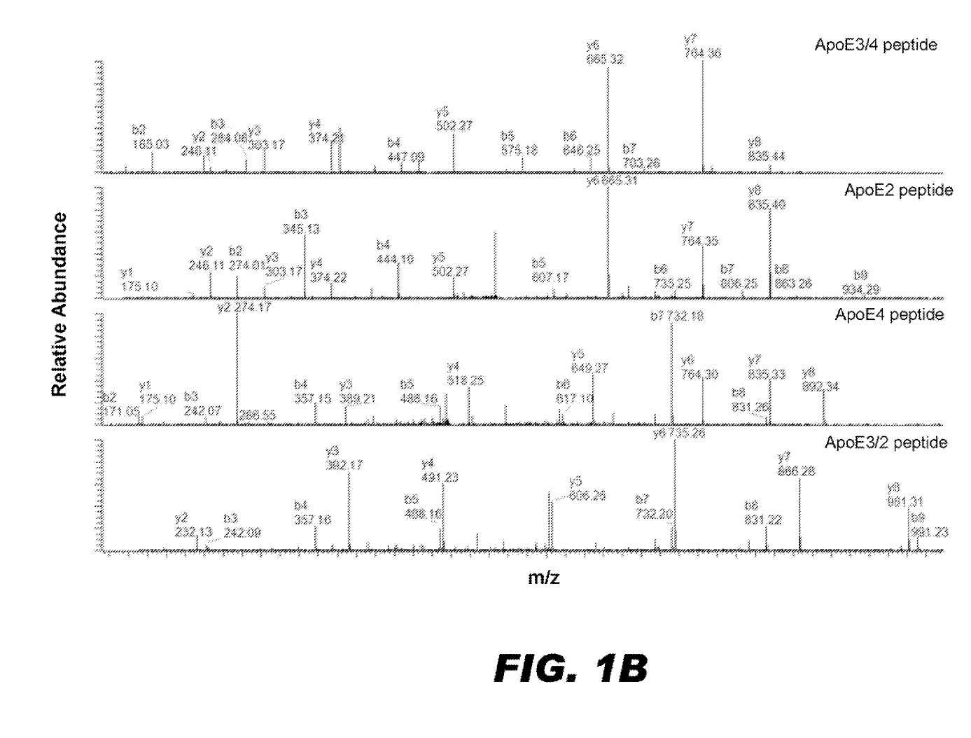

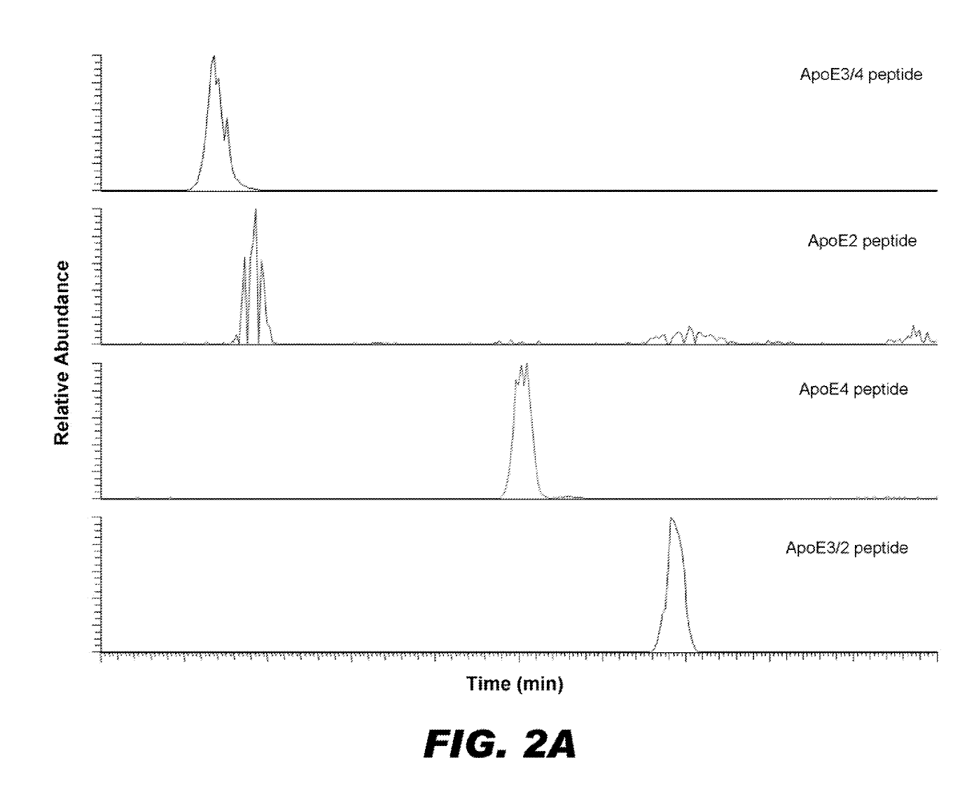

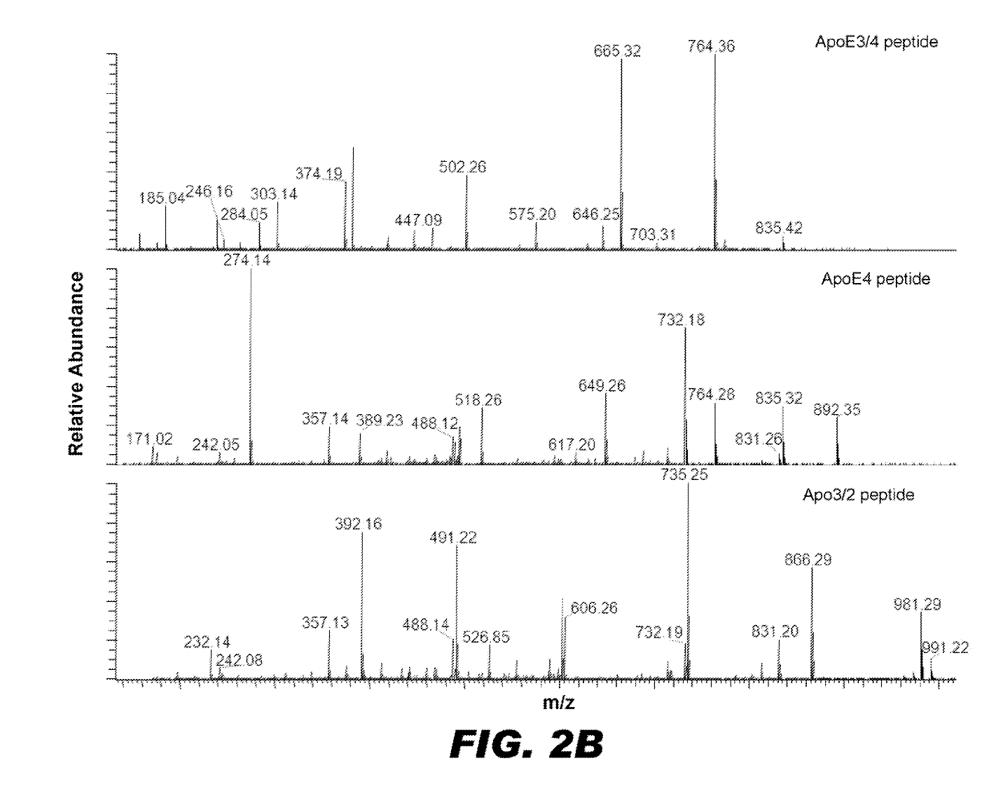

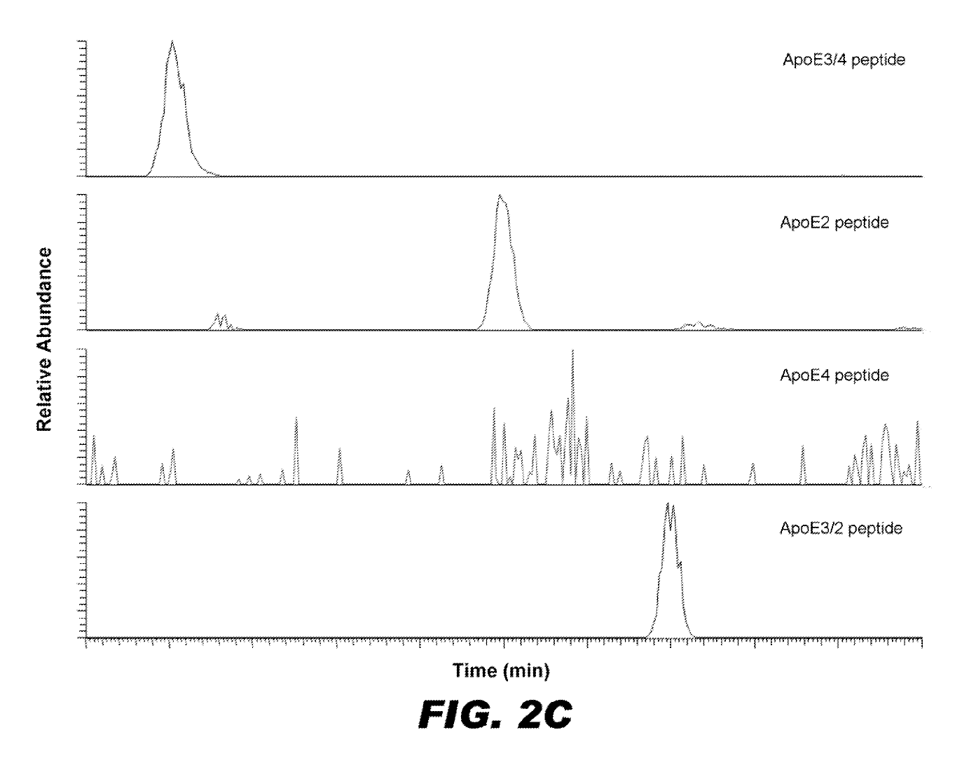

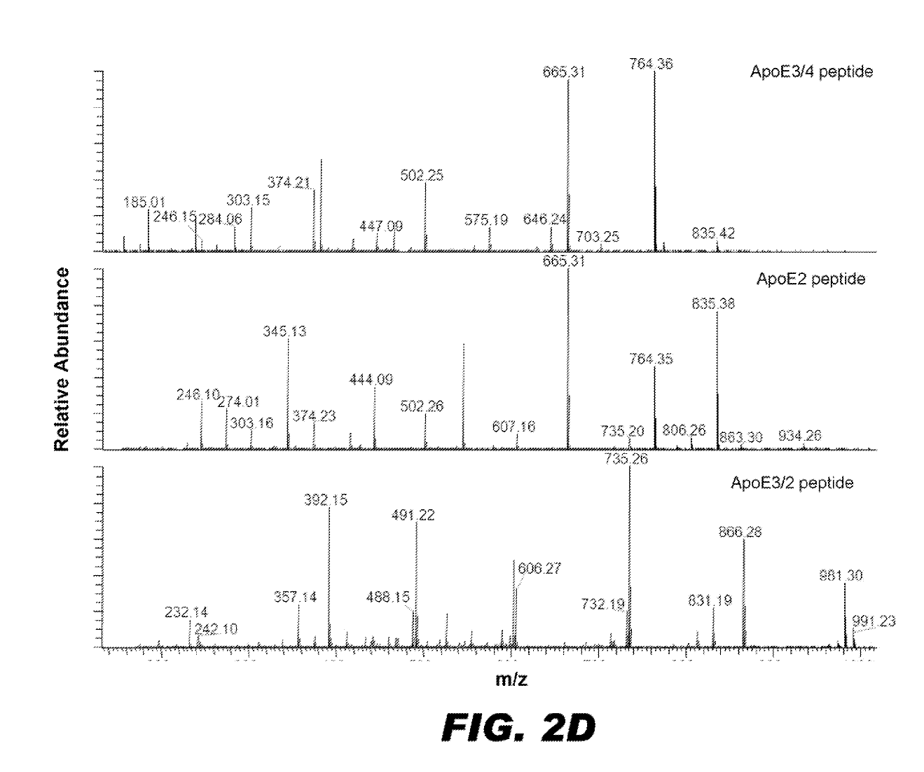

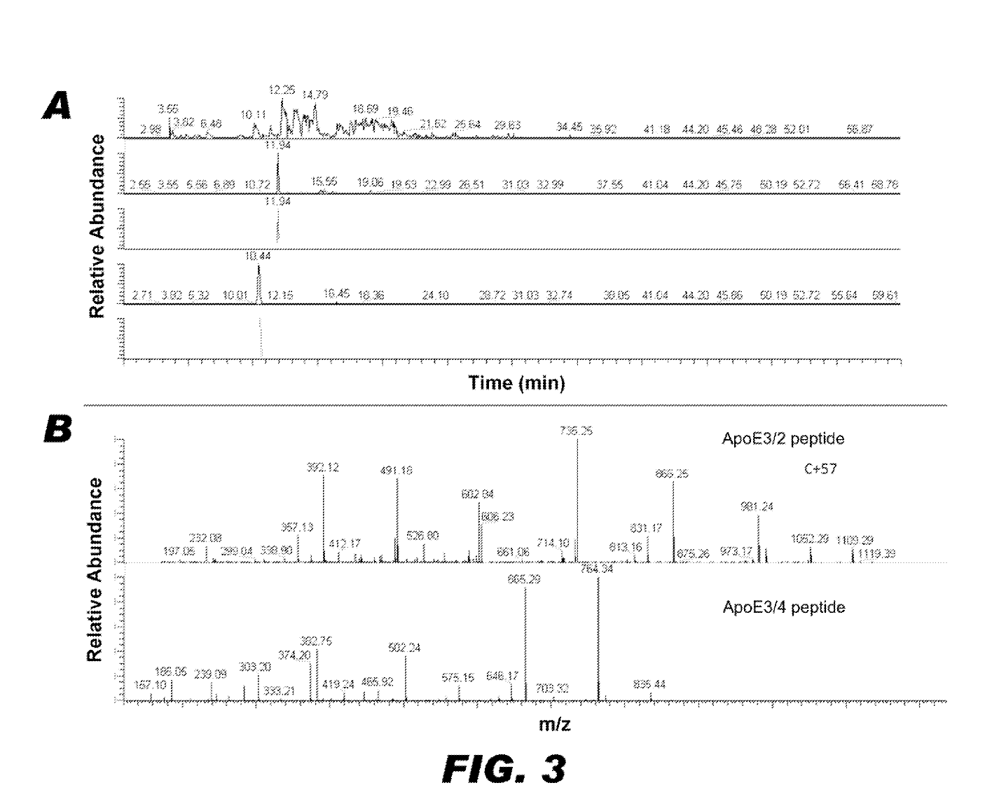

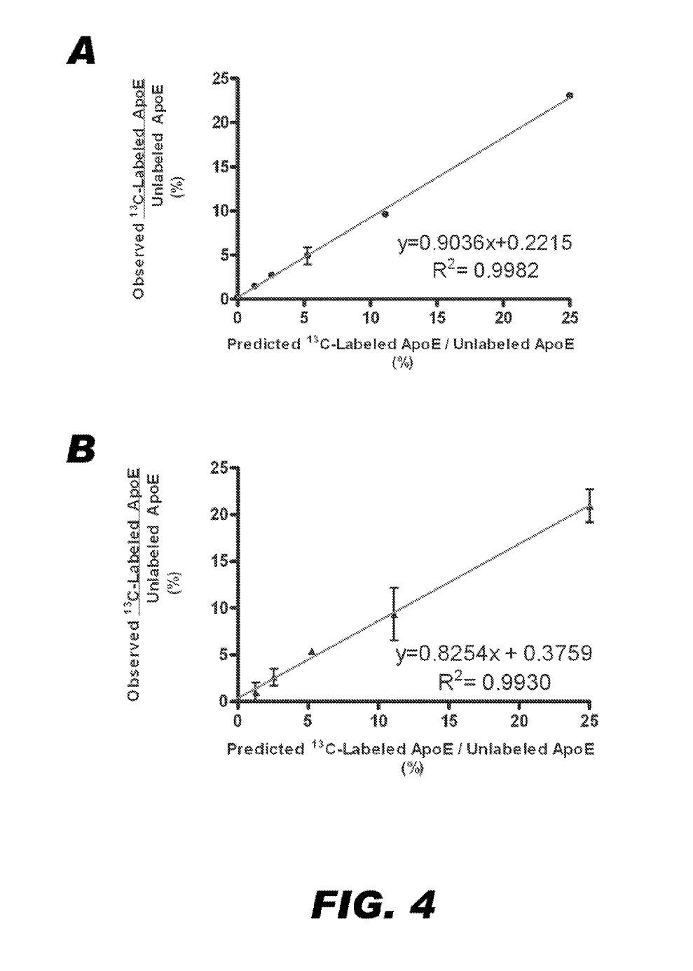

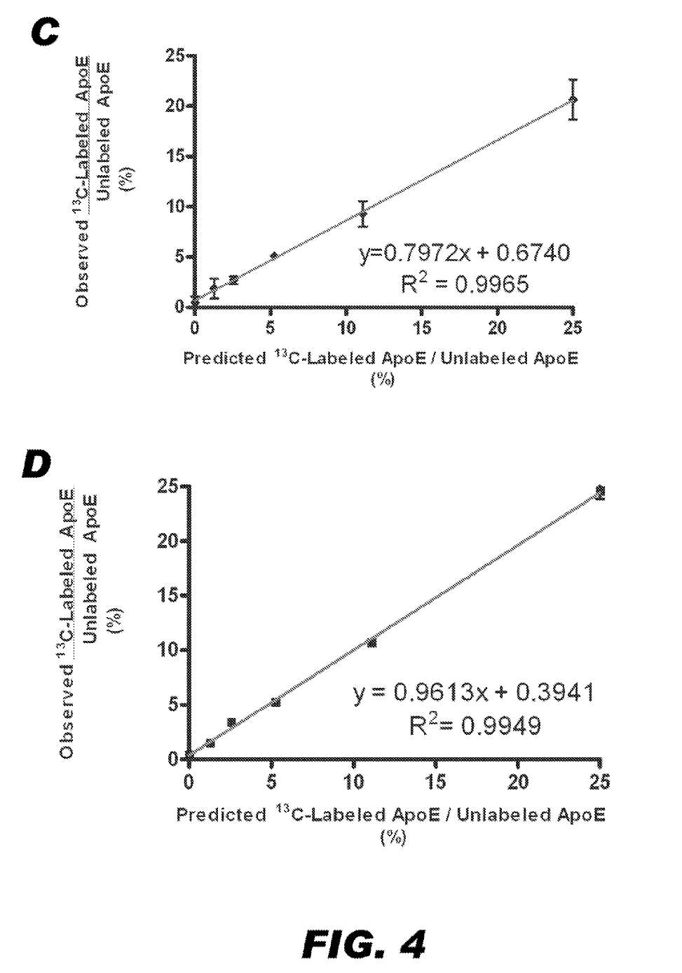

Four isotope specific ApoE peptides were detected (see Table 1). When pooled together, ApoE2 and ApoE4 media yielded all four isoform specific peptides (FIG. 1). Similarly, all four peptides were detected in CSF from heterozygous individuals expressing two different isoforms (FIG. 2). The ApoE peptides detected in FIGS. 1 and 2 were generated from ApoE proteins that were isolated by adsorption to Liposorb. FIG. 3 illustrates the detection of ApoE3 peptides that were generated from ApoE proteins that were isolated by immunoprecipitation. All four ApoE peptides showed strong linear correlations over the 1-20% ¹³C-labeling range with respect to the observed vs. predicted percent of labeled to unlabeled peptide (FIG. 4). These data indicate that the detection of these isoform specific peptides is robust and reproducible for quantitation.

TABLE 1
Human ApoE Isoform Specific Tryptic Peptides.

| Isoform | Peptide Sequence¹ | M (C + 57)² | SEQ ID NO: |
|---|---|---|---|
| E3&E2 | LGADMEDVCGR | 1221.5 | 1 |
| E3&E4 | LAVYQAGAR | 947.5 | 2 |
| E4 | LGADMEDVR | 1004.5 | 3 |
| E2 | CLAVYQAGAR | 1107.54 | 4 |

¹Bold residues indicate position of isoform variability (C₁₁₂ to R₁₁₂ or R₁₅₈ to C₁₅₈).
²Masses include alkylation of cysteine residues.

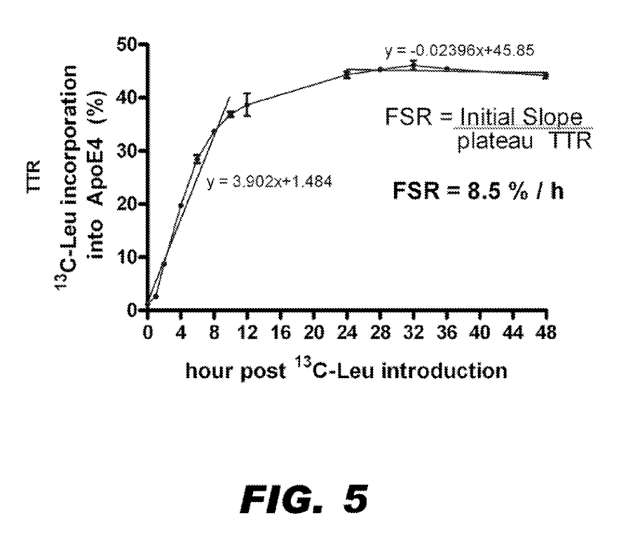

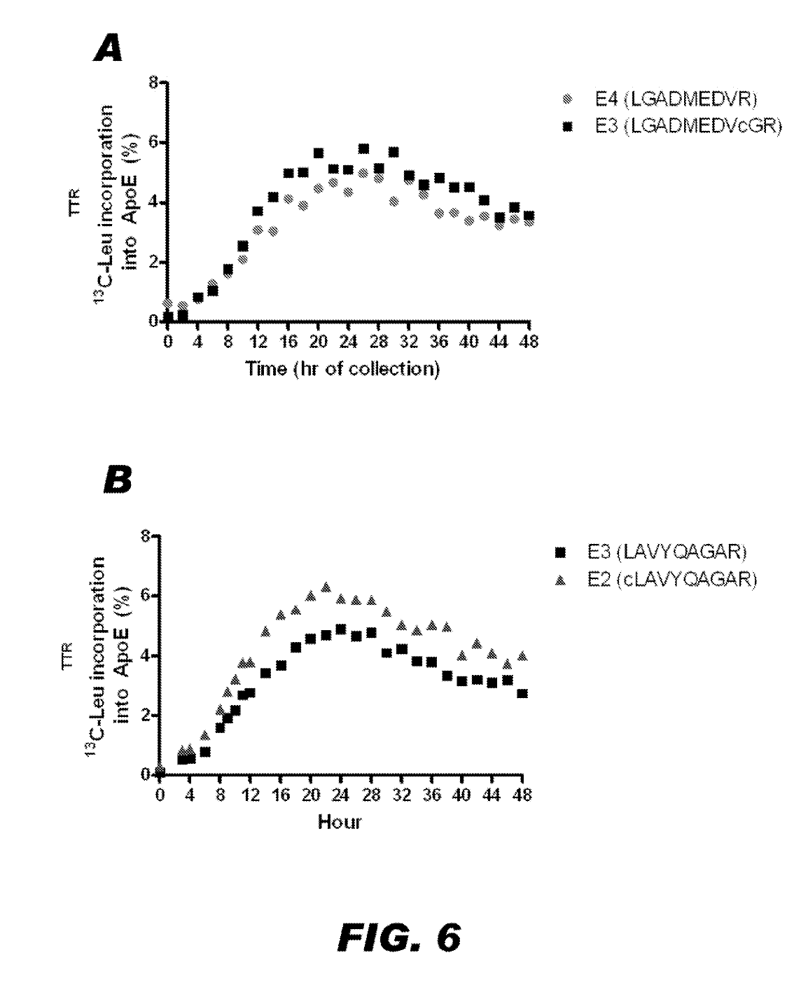

The metabolism of the ApoE specific isoforms was determined as detailed by Bateman et al. 2006, Nature Med 12(7):856-861. The fractional synthesis rate (FSR) of human ApoE4 in immortalized knock-in murine astrocytes is presented in FIG. 5. The FSR was calculated using the initial slope and the plateau tracer to tracee ration (TTR).

The data presented in this example reveal that four different isoform specific peptides of ApoE can be isolated, detected, and quantitated simultaneously in one sample.

### Example 2

### Quantitation of Soluble APP-alpha and APP-Beta in the Human Central Nervous System

In Alzheimer's disease, the protein Amyloid Precursor Protein (APP) has a central role in the pathogenesis of the disease. Amyloid-beta (Aβ) is one product of APP and is the main component of amyloid plaques, which are a hallmark of Alzheimer's disease. In addition, other metabolic products of APP include soluble APP (sAPP)-alpha and sAPP-beta which are products of the alpha-secretase pathway and beta-secretase, respectively. Digestion of APP with beta-secretase and gamma-secretase produces Aβ. Thus, the beta-secretase pathway generates Aβ and is an amyloidogenic pathway. Accordingly, the beta-secretase pathway is a major target to inhibit in Alzheimer's disease. Increasing the activity in the alpha-secretase pathway (i.e., the non-amyloidogenic pathway) has also been targeted as a potential treatment for Alzheimer's disease. The following example details a method that can quantify the alpha-secretase and beta-secretase pathways of APP in vivo.

The method enables the measurement of the production and clearance rates of two protein products (sAPP-alpha and sAPP-beta), after APP has been cleaved consecutively by alpha-secretase, or beta-secretase and gamma-secretase. This cleavage determines which pathway the APP will take, whether it becomes sAPP-alpha, which has been shown to be neuroprotective, or whether it goes down a pathway that will produce sAPP-beta and expose the membrane-bound APP C-terminal fragment to be cleaved by gamma-secretase, with an end result of amyloid-beta production.

APP was labeled with ¹³C₆-leucine by administration to human participants from 0-9 hours (Bateman et al., 2006 supra). CSF samples were collected by lumbar puncture every hour for a 36-hour or 48-hour time course. In addition, two human CSF samples without label (0%) were prepared. CHO cells were also incubated with 0, 1.25, 2.5, 5, 10, or 20% ¹³C₆-leucine and samples were taken at appropriate times. APP was immunoprecipitated using an antibody, 8E5 (Eli Lilly), which is not specific for either APP-alpha or APP-beta, but binds APP in general. APP samples were digested the endoproteinase ArgC or trypsin. The resultant peptides were analyzed by nano-LC tandem MS as detailed above. The labeled ion mass was shifted by either 6, 3, 2, and 1.5 m/z depending on the charge of any specific precursor (+1, +2, +3, and +4, respectively). From these data, the percent label of any given sample was determined.

Upon digestion, the APP specific peptides were sAPP-alpha (C-terminal or short) PGSGLTNIKTEEISEVKMDAEFR (SEQ ID NO:5) and sAPP-beta (C-terminal or short) PGSGLTNIKTEEISEVKM (SEQ ID NO:6). These two peptides were readily separated and quantified from a single biological sample. The labeled and unlabeled sAPP-alpha and sAPP-beta peptides signals were above the detection threshold, i.e., each produced reliable readings of the percent labeled leucine to the unlabeled in the media. Thus, several standard curves were plotted using the LC-MS results.

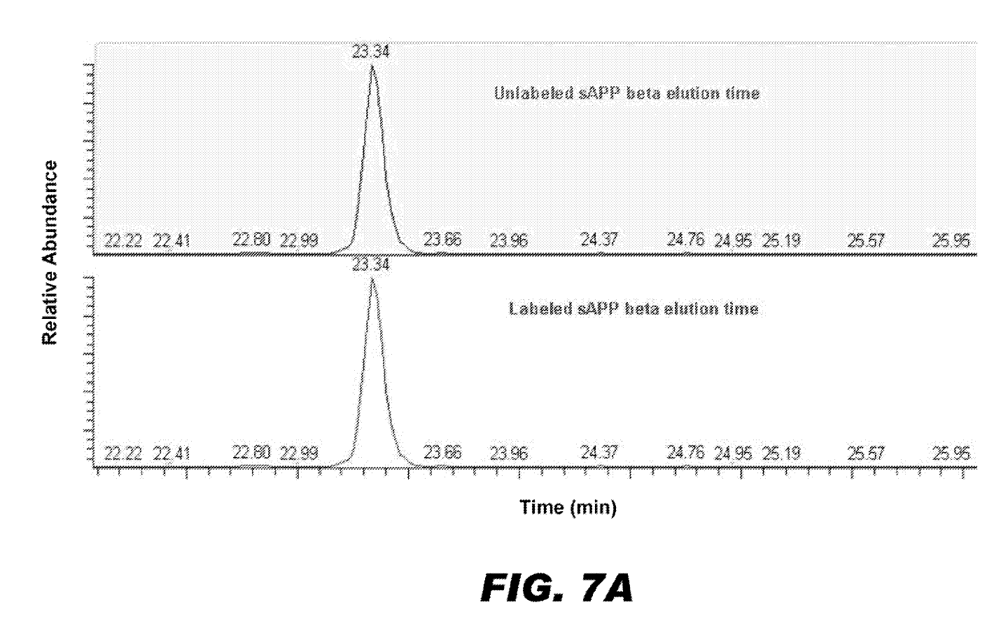

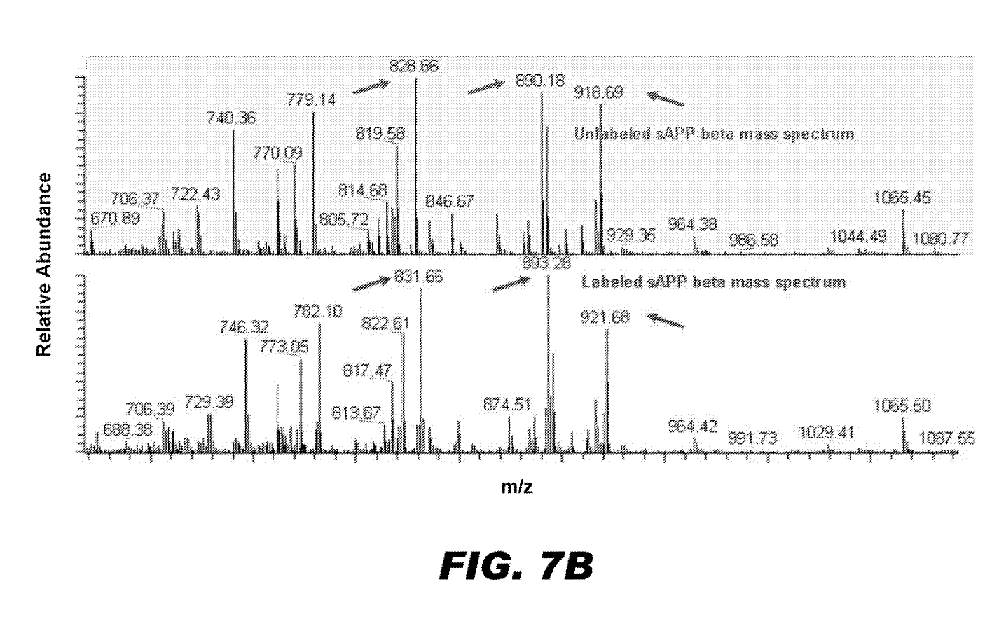

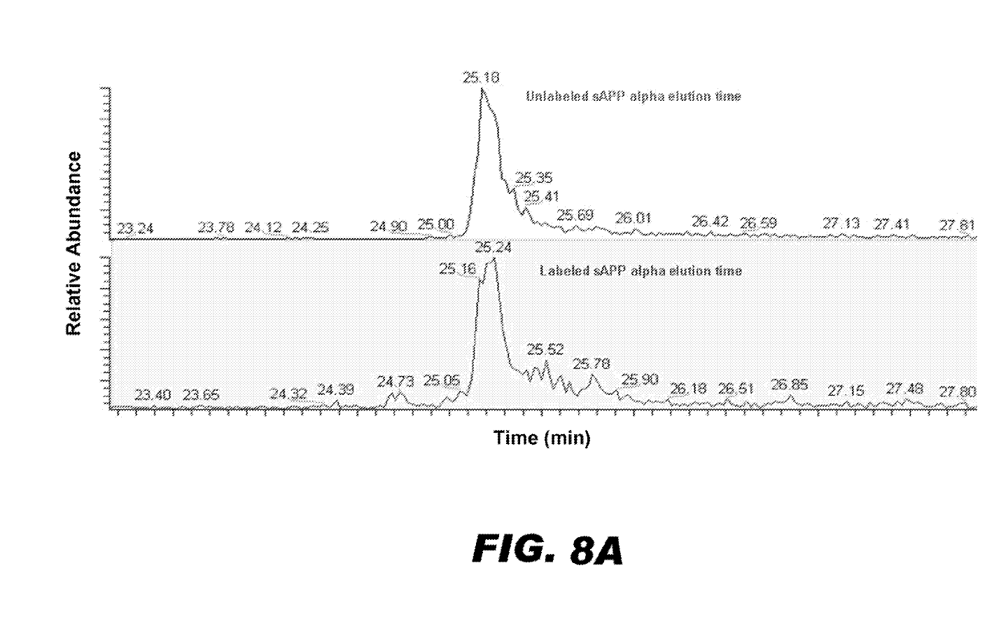

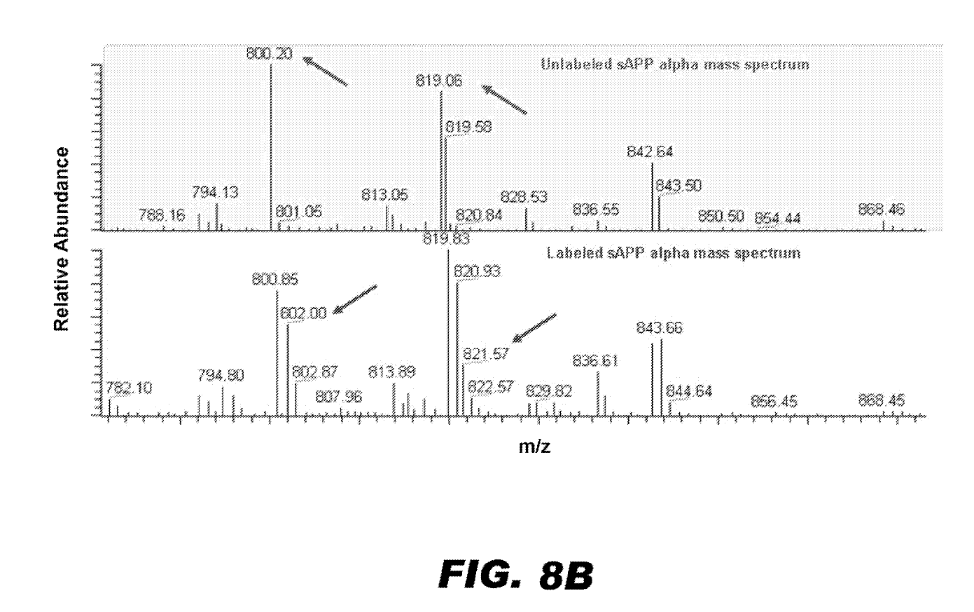

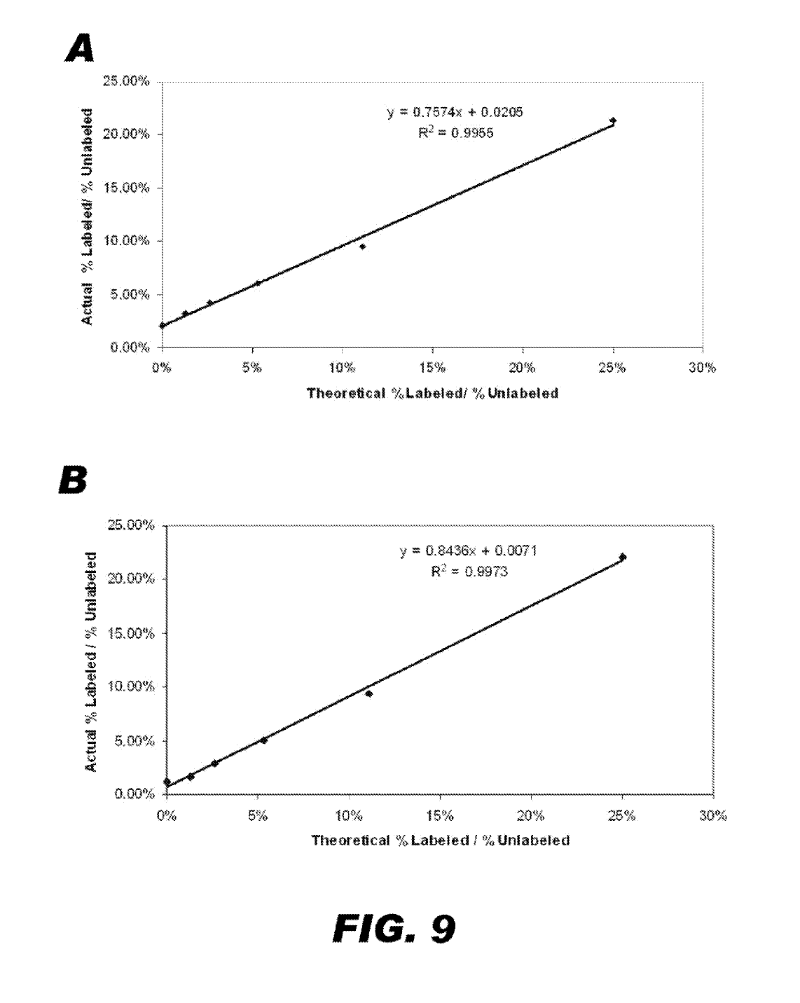

FIGS. 7 and 8 present the detection of sAPP-beta and sAPP-alpha, respectively, in a 20% leucine labeled CHO media sample. Standard curves comparing the theoretical percent of labeled to unlabeled peptide to the actual percent of labeled to unlabeled peptide show a linear relationship (FIG. 9) for each peptide.

The APP specific peptides were detected in CSF, and the fractional synthesis rate (FSR) and fractional clearance rate (FCR) of total sAPP were determined. The FSR (slope/precursor) was 3.9% per hour, and the FCR (slope) was 4.7% per hour (data not shown). The FSR and FCR of sAPP were approximately half of the corresponding rates of amyloid-beta.

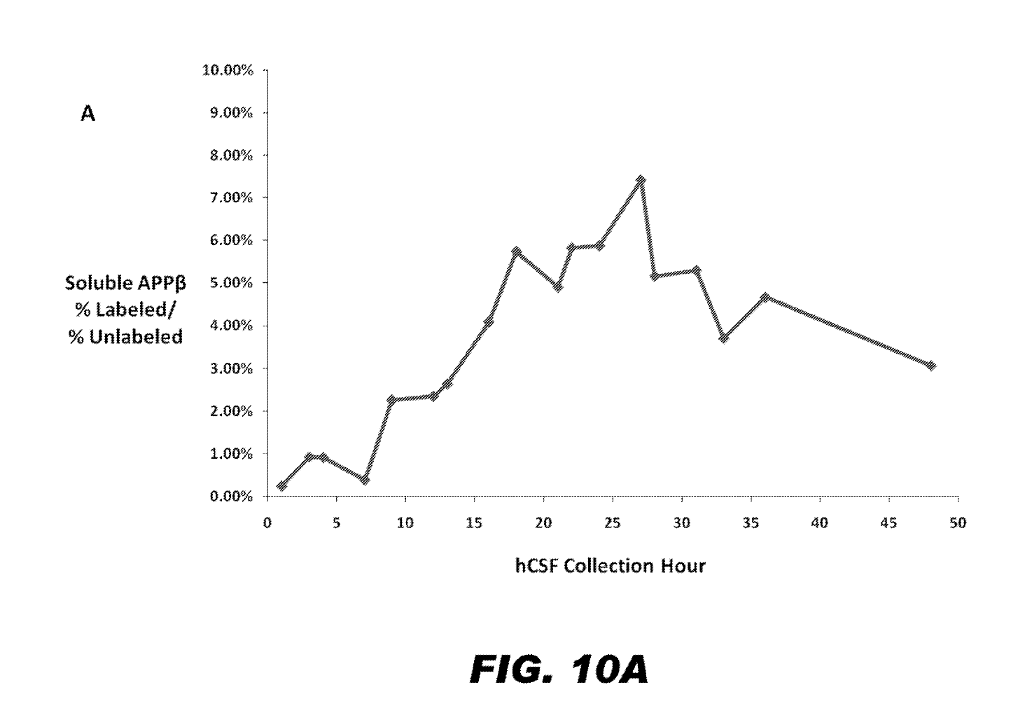

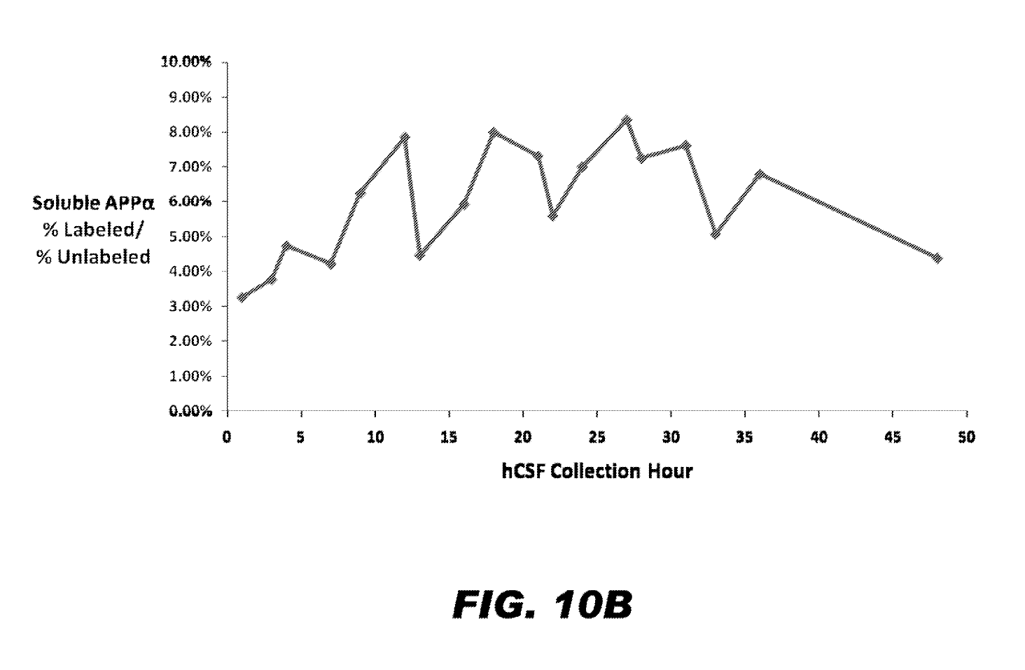

FIGS. 10(A) and (B) present the change in ratio of the percent labeled to percent unlabelled soluble APP alpha (FIG. 10(B)) and beta (FIG. 10(A)) in a human subject, as measured in CSF over 48 hours. The production and clearance rates of soluble APP alpha and soluble APP beta can be determined by calculating the production and clearance rates of the summated APP alpha or APP beta peptide ions.

These data demonstrate that two different digestion products of APP can be detected and quantitated simultaneously in one sample.

### Example 3

### Simultaneous Detection and Quantitation of Amyloid-Beta Isoforms

Amyloid-beta (Aβ) is generated from APP by digestion with gamma- and beta-secretases. Several different forms of Aβ exist that differ at the C-terminal end (e.g., Aβ₁₋₃₈, Aβ₁₋₄₀, and Aβ₁₋₄₂). Understanding the metabolism of each of these isoforms of Aβ may provide insight into the pathophysiology of Alzheimer's disease and may be highly useful for therapeutic development as it is believed that decreasing Aβ₁₋₄₂ selectively or increasing Aβ₁₋₃₈ or smaller species will be beneficial in the treatment of Alzheimer's disease.

Digestion of Aβ with peptidases will generate unique peptides, including unique C-terminal and, potentially, N-terminal peptides. For example, when Aβ is digested with trypsin, the C-terminal peptide sequence for Aβ₂₉₋₃₈ is GAIIGLMVGG (SEQ ID NO:7), for Aβ₂₉₋₄₀ is GAIIGLMVGGVV (SEQ ID NO:8), and for Aβ₂₉₋₄₂ is GAIIGLMVGGVVIA (SEQ ID NO:9).

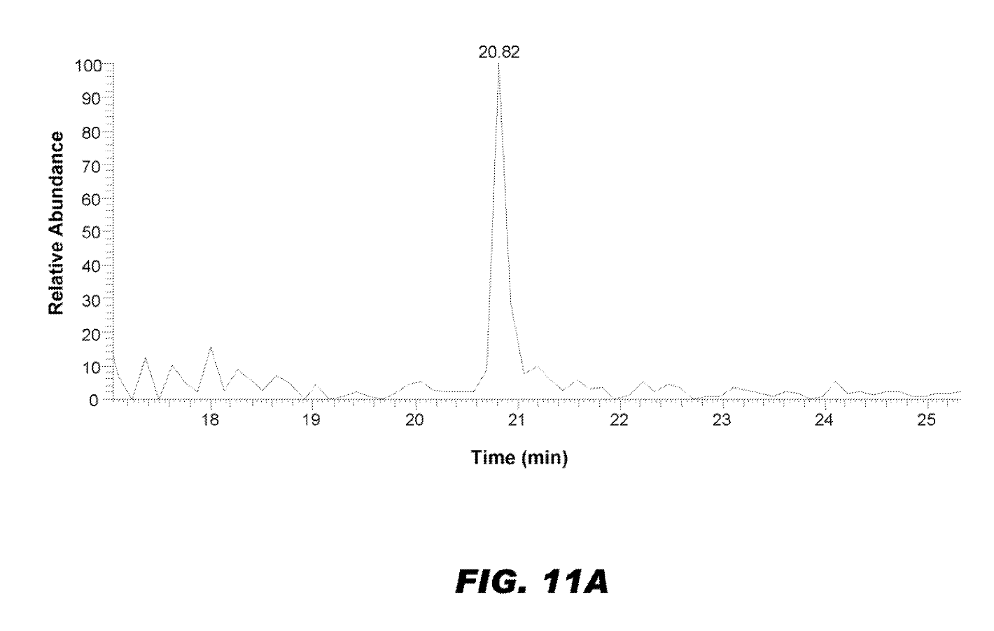

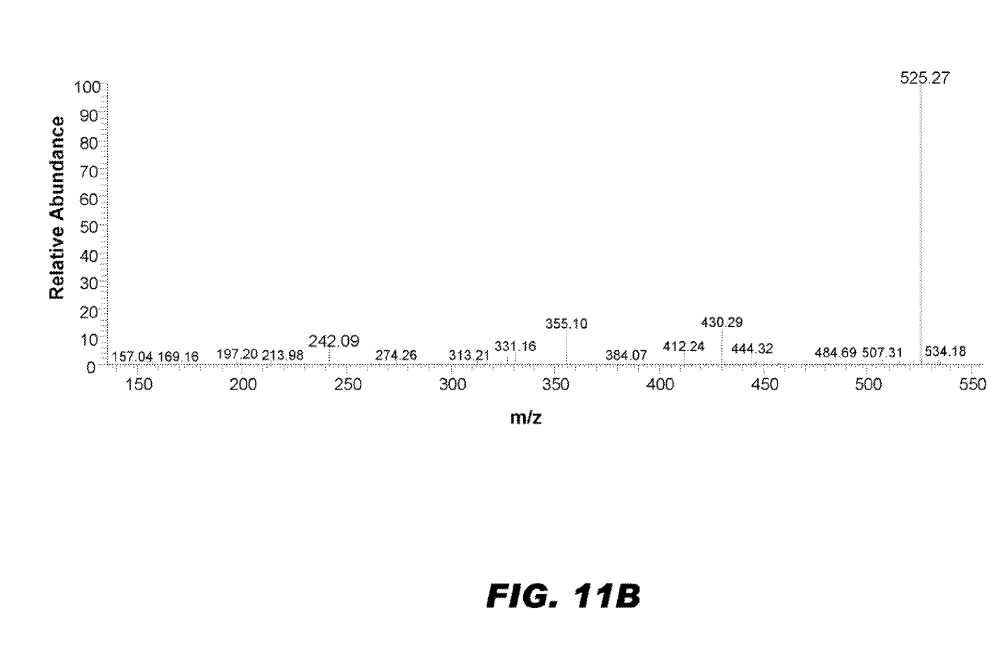

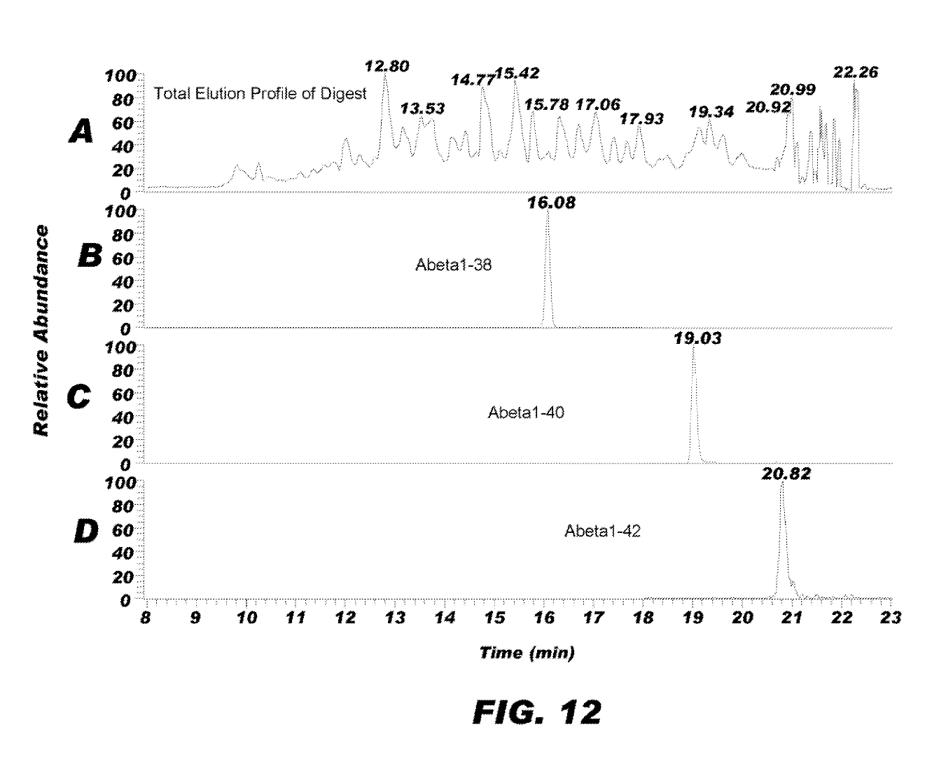

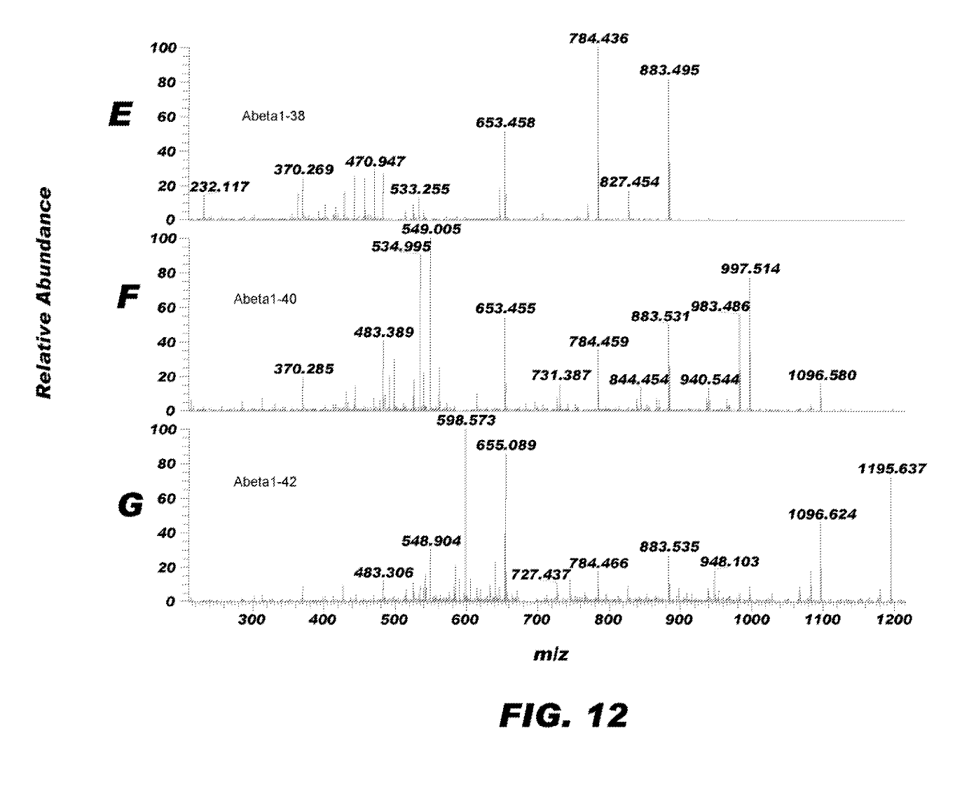

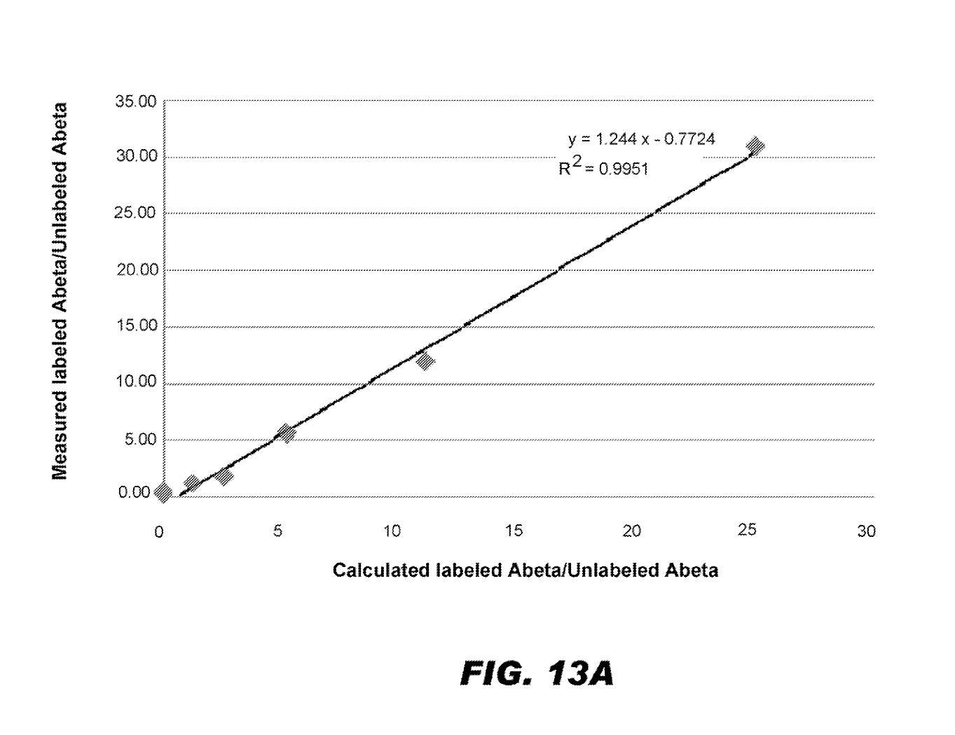

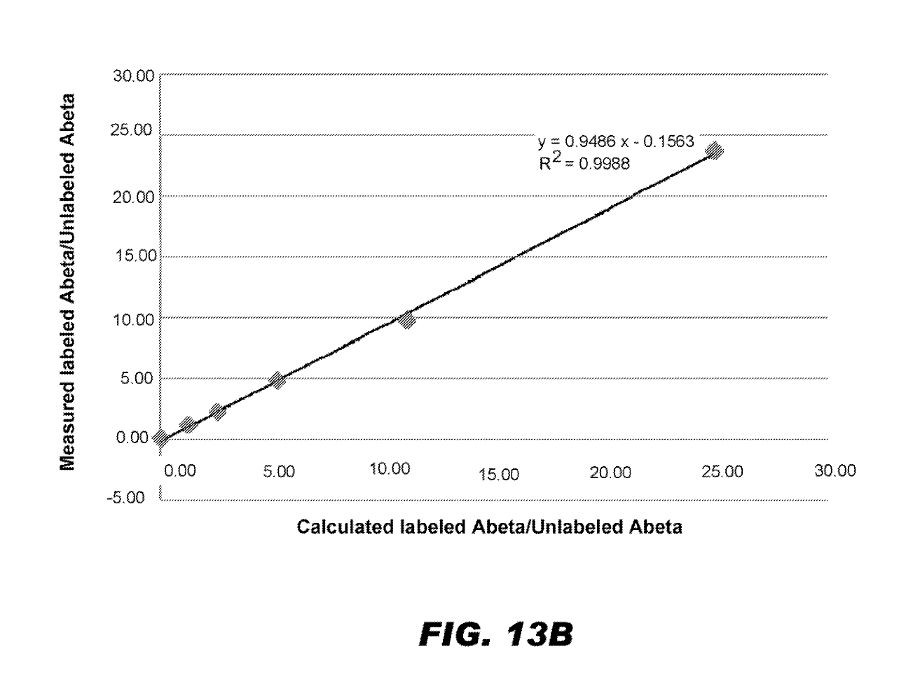

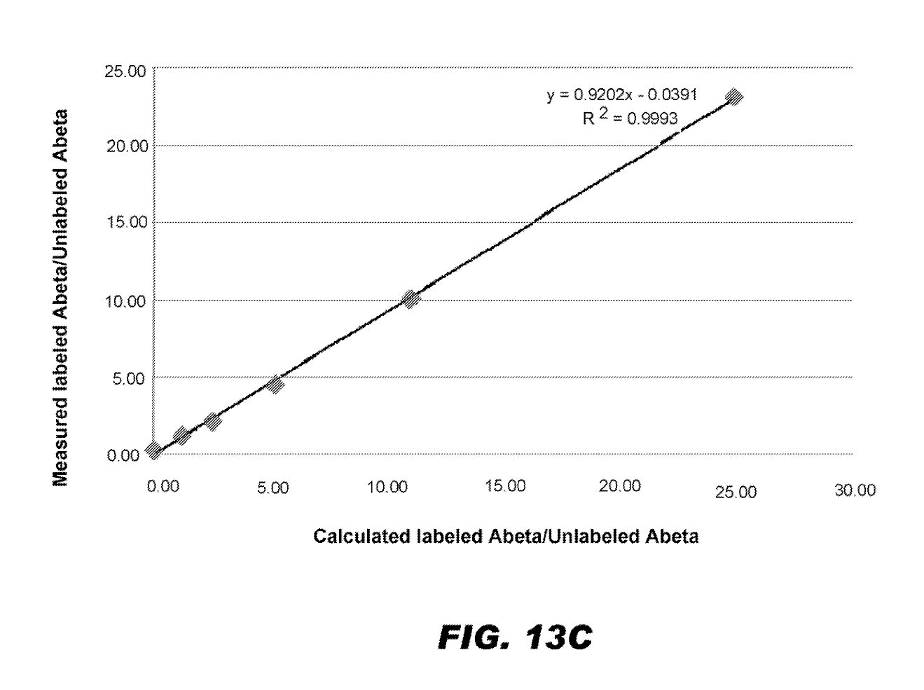

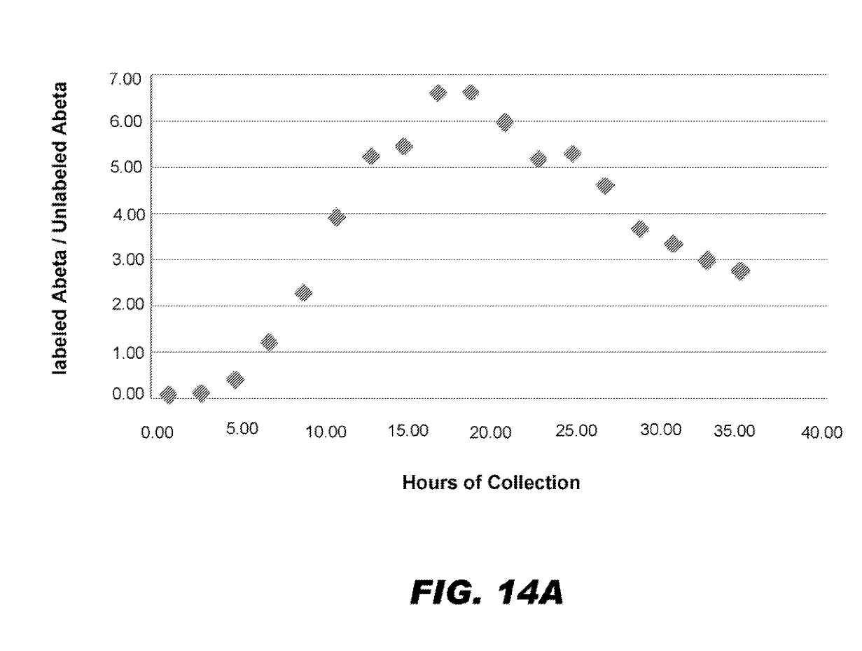

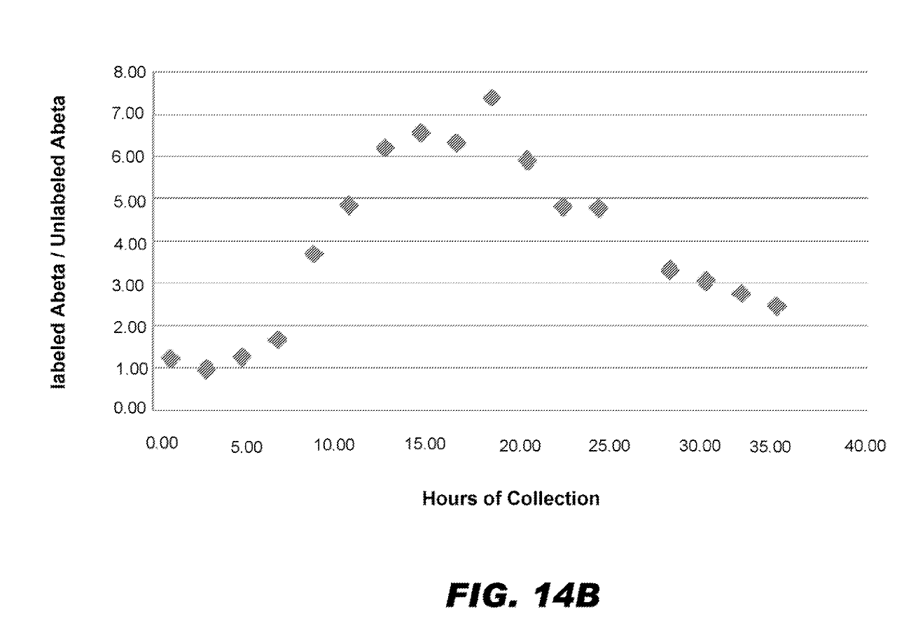

Labeled and unlabeled Aβ may be isolated from CSF and cell culture media as detailed above. Aβ may be immunoprecipitated by N-terminal or mid-domain binding antibodies, and then digested with trypsin (or another peptidase). For example, Aβ was immunoprecipitated from media with HJ5.1 anti-Aβ antibody (mid-domain) and trypsin digested. The resultant peptide (Aβ₂₉₋₄₀) was analyzed by nano-LC tandem MS as detailed above. The results are presented in FIG. 11. Additionally, two or more different C-terminal Aβ peptides were separated on an Xbridge C8 (Waters Corporation) liquid chromatography column and by their different masses with mass spectrometry. Alternatively, Aβ may be digested with Lys-N and then analyzed, as illustrated by FIGS. 12-14.

1. A method for simultaneously measuring the in vivo metabolism of two or more isoforms of a biomolecule produced in the central nervous system of a subject, the method comprising:
a) administering a labeled moiety to the subject, the labeled moiety being incorporated into the biomolecule as the biomolecule is produced in the subject;
b) obtaining a biological sample from the subject, the biological sample comprising a first biomolecule fraction labeled with the moiety, and a second biomolecule fraction not labeled with the moiety, the first biomolecule fraction comprising two or more labeled isoforms of the biomolecule, and the second biomolecule fraction comprising two or more unlabeled isoforms of the biomolecule; and
c) detecting the amount of each labeled isoform and the amount of each unlabeled isoform, wherein the ratio of labeled isoform to unlabeled isoform for a particular isoform is directly proportional to the metabolism of the particular isoform in the subject.

2. The method of claim 1, wherein the biomolecule is a protein, the two or more isoforms of the protein being selected from the group consisting of cleavage fragments generated in vivo by protease activity, cleavage fragments generated in vivo by non-protease activity, genetic alleles of the protein, RNA processing products, and posttranslational products.

3. The method of claim 2, wherein the genetic alleles, RNA processing products, and posttranslational products are detected in step (c) as isoform specific fragments generated ex vivo by protease activity.

4. The method of claim 2, wherein the protein is selected from the group consisting of amyloid-beta, apolipoprotein E, apolipoprotein J, synuclein, soluble amyloid precursor protein, Tau, TDP-43, huntingtin, progranulin, alpha-2 macroglobulin, S100B, myelin basic protein, an interleukin, and TNF.

5.-8. (canceled)

9. The method of claim 1, further comprising isolating the labeled isoforms and the unlabeled isoforms from the biological sample.

10. The method of claim 9, wherein the labeled fragments and the unlabeled fragments are isolated from the biological sample by a method selected from the group consisting of immunoprecipitation, adsorption to a derivatized polymer, liquid chromatography, and combinations thereof.

11. (canceled)

12. The method of claim 1, wherein the biomolecule is a protein; the labeled moiety is ¹³C₆-leucine; the protein is apolipoprotein E (ApoE); ApoE is isolated from the biological sample by immunoprecipitation or adsorption to a derivatized polyhydroxymethylene polymer; the isoforms that are detected are ApoE isoform specific fragments generated by ex vivo cleavage of ApoE; and the labeled and unlabeled isoforms are detected by mass spectrometry.

13. The method of claim 1, wherein the biomolecule is a protein; the labeled moiety is ¹³C₆-leucine; the protein is amyloid precursor protein (APP); the isoforms are soluble APP-alpha and soluble APP-beta; the labeled and unlabeled isoforms are isolated from the sample by immunoprecipitation; and the labeled and unlabeled isoforms are detected by mass spectrometry.

14. The method of claim 1, wherein the biomolecule is a protein; the labeled moiety is ¹³C₆-leucine; the protein is amyloid-beta; the isoforms are selected from the group consisting of amyloid-beta₁₋₃₈, amyloid-beta₁₋₃₉, amyloid-beta₁₋₄₀, amyloid-beta₁₋₄₁, and amyloid-beta₁₋₄₂; the labeled and unlabeled isoforms are isolated from the sample by immunoprecipitation; and the labeled and unlabeled isoforms are detected by mass spectrometry.

15. A method for determining whether a therapeutic agent affects the in vivo metabolism of two or more isoforms of a biomolecule produced in the central nervous system of a subject, the method comprising:
d) administering the therapeutic agent to the subject;
e) administering a labeled moiety to the subject, the labeled moiety being incorporated into the biomolecule as the biomolecule is produced in the subject;
f) obtaining a biological sample from the subject, the biological sample comprising a first biomolecule fraction labeled with the moiety, and a second biomolecule fraction not labeled with the moiety, the first biomolecule fraction comprising two or more labeled isoforms of the biomolecule, and the second biomolecule fraction comprising two or more unlabeled isoforms of the biomolecule;
g) detecting the amount of each labeled isoform and the amount of each unlabeled isoform in the biological sample, wherein the ratio of labeled isoform to unlabeled isoform for a particular isoform is directly proportional to the metabolism of the particular isoform in the subject; and
h) comparing the metabolism of each isoform to a suitable control value, such that a change from the control value for a particular isoform indicates the therapeutic agent affects the metabolism of the particular isoform in the central nervous system of the subject.

16. The method of claim 15, wherein step (b) is performed before step (a).

17. The method of claim 15, wherein the biomolecule is a protein, the two or more isoforms of the protein being selected from the group consisting of cleavage fragments generated in vivo by protease activity, cleavage fragments generated in vivo by non-protease activity, genetic alleles of the protein, RNA processing products, and posttranslational products.

18. The method of claim 17, wherein the genetic alleles, RNA processing products, and posttranslational products are detected in step (d) as isoform specific fragments generated ex vivo by protease activity.

19. The method of claim 17, wherein the protein is selected from the group consisting of amyloid-beta, apolipoprotein E, apolipoprotein J, synuclein, soluble amyloid precursor protein, Tau, TDP-43, huntingtin, progranulin, alpha-2 macroglobulin, S100B, myelin basic protein, an interleukin, and TNF.

20.-24. (canceled)

25. The method of claim 15, wherein the suitable control value is selected from the group consisting of the same subject prior to administration of the therapeutic agent, a control subject who is not administered the therapeutic agent, and a control isoform.

26. The method of claim 15, further comprising isolating the labeled isoforms and the unlabeled isoforms from the biological sample.

27. The method of claim 26, wherein the labeled isoforms and the unlabeled isoforms are isolated from the biological sample by a method selected from the group consisting of immunoprecipitation, adsorption to a derivatized polymer, liquid chromatography, and combinations thereof.

28. (canceled)

29. The method of claim 15, wherein the biomolecule is a protein; the labeled moiety is ¹³C₆-leucine; the protein is apolipoprotein E (ApoE); ApoE is isolated from the biological sample by immunoprecipitation or adsorption to a derivatized polyhydroxymethylene polymer; the isoforms that are detected are ApoE isoform specific fragments generated by ex vivo cleavage of ApoE; and the labeled and unlabeled isoforms are detected by mass spectrometry.

30. The method of claim 15, wherein the biomolecule is a protein; the labeled moiety is ¹³C₆-leucine; the protein is amyloid precursor protein (APP); the isoforms are soluble APP-alpha and soluble APP-beta; the labeled and unlabeled isoforms are isolated from the sample by immunoprecipitation; and the labeled and unlabeled isoforms are detected by mass spectrometry.

31. The method of claim 15, wherein the biomolecule is a protein; the protein is amyloid-beta; the labeled moiety is ¹³C₆-leucine; the isoforms are selected from the group consisting of amyloid-beta₁₋₃₈, amyloid-beta₁₋₃₉, amyloid-beta₁₋₄₀, amyloid-beta₁₋₄₁, and amyloid-beta₁₋₄₂; the labeled and unlabeled isoforms are isolated from the sample by immunoprecipitation; and the labeled and unlabeled isoforms are detected by mass spectrometry.
